(19) United States
(12) Patent Application Publication
(10) Pub. No.: US 2026/0183435 A1
(43) Pub. Date: Jul. 2, 2026

(54) RADIOACTIVE EVANS BLUE DERIVATIVE PHARMACEUTICAL AQUEOUS SOLUTION, PREPARATION METHOD THEREFOR, AND USE THEREOF

(71) Applicant: SINOTAU PHARMACEUTICAL GROUP, Beijing (CN)

(72) Inventors: Guogui YANG, Beijing (CN); Jin HAO, Beijing (CN); Zetian DU, Beijing (CN); Jiale TIAN, Beijing (CN)

(73) Assignee: SINOTAU PHARMACEUTICAL GROUP, Beijing (CN)

(21) Appl. No.: 18/858,013

(22) PCT Filed: Jun. 20, 2023
(86) PCT No.: PCT/CN2023/101452

§ 371 Date: Oct. 18, 2024

(30) Foreign Application Priority Data
Apr. 20, 2022 (CN) 202210416293.6
Apr. 20, 2022 (CN) 202210416297.4

(51) Int. Cl. A61K 51/08; A61K 47/12; A61K 47/18; A61K 47/20; A61K 47/26; A61K 51/04; C07B 59/00

(52) U.S. Cl. CPC A61K 51/088; A61K 47/12; A61K 47/18; A61K 47/20; A61K 47/26; A61K 51/0446; A61K 51/0459; A61K 51/0482; A61K 51/0497; A61K 51/083; C07B 59/002; C07B 59/008; A61K 2121/00; C07B 2200/05

(57) ABSTRACT

The present application relates to a radiopharmaceutical aqueous solution and a use thereof. The radiopharmaceutical aqueous solution comprises a complex formed by a targeting molecule modified by an Evans blue (EB) fragment and a radioactive metal nuclide, as well as a stabilizer, the stabilizer preferably being one or two or more among gentisic acid, ethanol, and methionine.

### TECHNICAL FIELD

The present application relates to a radiopharmaceutical aqueous solution with high chemical stability and high radiochemical stability and a preparation method therefor, and in particular to a radionuclide complex modified by an Evans blue (EB) fragment.

### BACKGROUND ART

The circulation half-life of drug molecules in the body can greatly affect the effectiveness and availability of the drug, especially for radiolabeled diagnostic and/or therapeutic drugs. Radiopharmaceuticals need a sufficiently long half-life in the body to allow the radiopharmaceuticals to fully bind to the target, and be gradually cleared from other non-target tissues or organs, so as to achieve their expected diagnostic and/or therapeutic effects. In a method for extending the in vivo half-life of small molecule drugs, structural modification of small molecules to enable them to reversibly bind to human serum albumin (HSA) in vivo is a commonly used method. Evans blue is an azo dye that is commonly used to measure blood-brain barrier integrity, vascular permeability, blood volume, and cell activity, due to its high affinity for serum albumin. The structural fragments of Evans blue dye molecules are used to modify the structure of targeting molecules (such as DOTATATE, PSMA-617, etc.). The resulting Evans blue derivative molecules, such as DOTA-EB-TATE, EB-PSMA, etc., can reversibly bind to endogenous serum albumin through the molecules, and serum albumin is used as a reversible carrier of drug molecules to prolong the half-life of drug molecules in the blood. It was reported in a literature (Bioconjugate Chem. 2018, 29, 3213-3221) that, Evans blue derivatives have a longer in vivo circulation half-life, a higher tumor uptake rate, and a longer retention time in the tumor. Moreover, due to their extended in vivo half-life, they can further improve drug efficacy, reduce dosage and frequency of administration, and reduce drug toxicity.

The structural formula of Evans blue dye is represented by the following Formula VI:

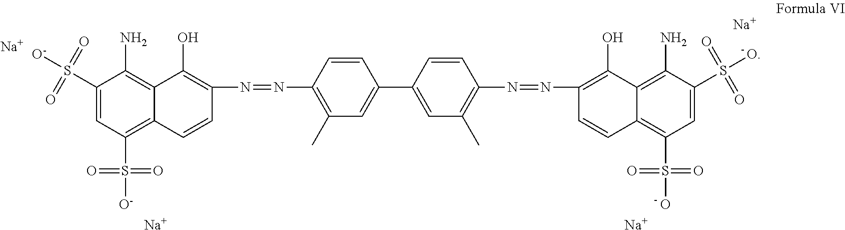

The relative molecular weight of the Evans blue fragment and the linker introduced into a drug molecular structure is relatively large, which will greatly affect the physical and chemical properties of the molecule, making the pharmaceutical aqueous solution comprising Evans blue derivatives face more challenges in the formulation process research. For example, in the drug PLUVICTO™ (lutetium Lu 177 vipivotide tetraxetan) injection, which is approved for treatment of PSMA-positive metastatic castration-resistant prostate cancer (mCRPC), the active pharmaceutical ingredient (API) is [¹⁷⁷Lu]Lu-PSMA-617, and the stabilizers are 0.39 mg/mL gentisic acid, and 50.0 mg/mL sodium ascorbate. After structural modification of [¹⁷⁷Lu]Lu-PSMA-617 with Evans blue fragments, the stability properties of the resulting molecule [¹⁷⁷Lu]Lu-EB-PSMA are completely different from those of [¹⁷⁷Lu]Lu-PSMA-617. The addition of ascorbic acid and a salt thereof not only fails to play the role of stabilizer, but also accelerates the radiolysis of [¹⁷⁷Lu]Lu-EB-PSMA.

Currently, no radiopharmaceuticals comprising Evans blue derivatives have been approved for marketing for radiotherapy and/or diagnosis of tumors. Therefore, there is a need to develop a stable formulation and a suitable production process for pharmaceutical aqueous solutions comprising Evans blue derivatives.

### SUMMARY

The object of the present application is to provide a radiopharmaceutical aqueous solution, which comprises a complex formed by a targeting molecule modified with an Evans blue (EB) fragment and a radioactive metal nuclide, as well as a stabilizer. Furthermore, the present application also provides a method for preparing a radiopharmaceutical aqueous solution. Particularly, the present application relates to the following technical solutions:
1. A radiopharmaceutical aqueous solution, comprising: a complex formed by a compound represented by Formula I or a pharmaceutically acceptable ester, amide, solvate or salt thereof, or a compound represented by Formula I or a salt of a pharmaceutically acceptable ester thereof, or a compound represented by Formula I or a salt of a pharmaceutically acceptable amide thereof, or a compound represented by Formula I or a solvate of a pharmaceutically acceptable ester thereof, or a compound represented by Formula I or a solvate of a pharmaceutically acceptable amide thereof, or a compound represented by Formula I or a solvate of a pharmaceutically acceptable salt thereof, and a radioactive metal nuclide, as well as a stabilizer which is present at a total concentration of 0.5-400 mg/mL,

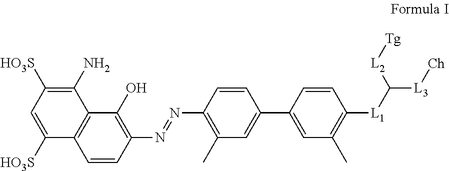

wherein
L₁ is —(CH₂)ₘ—, wherein m is an integer from 0 to 12, wherein each CH₂ may be individually replaced with —O—, —NH(CO)— or —(CO) NH—, providing no two adjacent CH₂ groups are replaced;
L₂ is a C₁-C₆₀ linking group, optionally including —O—, —S—, —S(O)—, —S(O)₂—, —N(R)—, —C(═O)—, —C(═O)O—, —OC(═O)—, —N(R)C(═O)—, —C(═O)N(R)—, —OC(═O)O—, —N(R)C(═O)O—, —OC(═O)N(R)—,

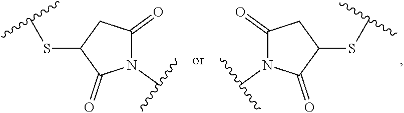

wherein each R is H or C₁-C₆ alkyl;
L₃ is —(CH₂)ₙ—, wherein n is an integer from 0 to 12, wherein each CH₂ may be individually replaced with —O—, —NH(CO)— or —(CO)NH—, providing no two adjacent CH₂ groups are replaced;
Ch is a chelating group; and
Tg is a targeting group.
2. The pharmaceutical aqueous solution according to item 1, wherein L₁ is —NH(CO)—, and L₃ is —NH(CO)CH₂—.
3. The pharmaceutical aqueous solution according to item 1, wherein Ch is selected from:

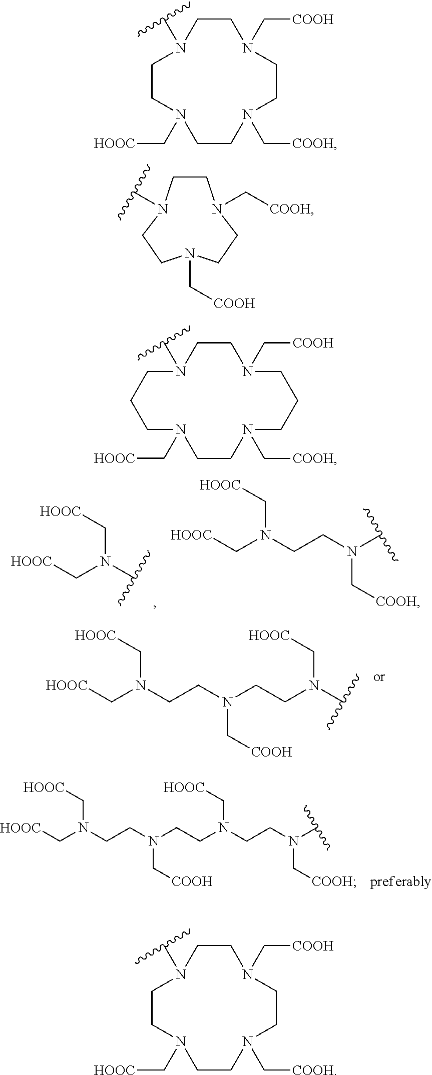

4. The pharmaceutical aqueous solution according to item 1, wherein Tg is selected from a chemical group capable of targeting somatostatin receptor (SSTR), prostate-specific membrane antigen (PSMA), fibroblast activation protein (FAP), folate receptor (FR), epidermal growth factor receptor or integrin.
5. The pharmaceutical aqueous solution according to item 4, wherein Tg is selected from:

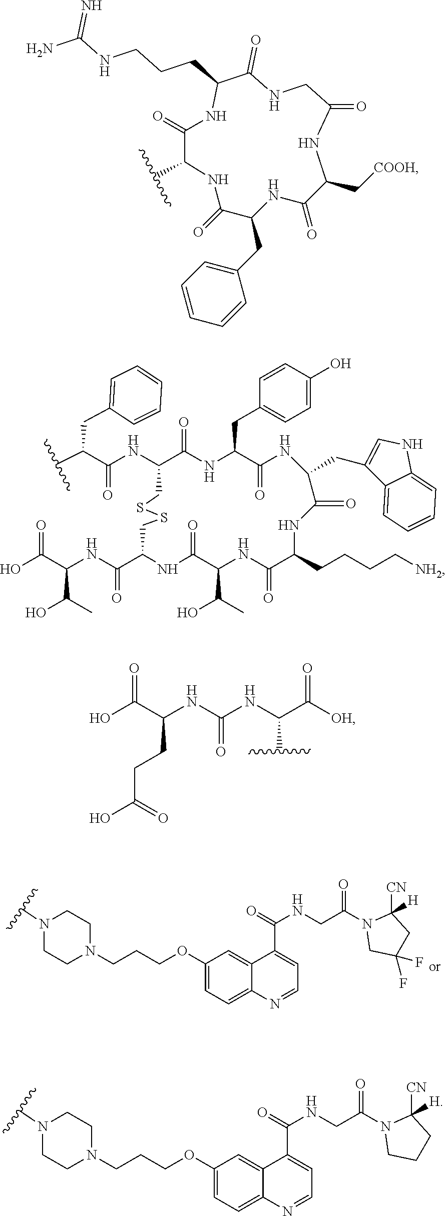

6. The pharmaceutical aqueous solution according to item 1, wherein Formula I is Formula II, Formula III, Formula IV or Formula V,

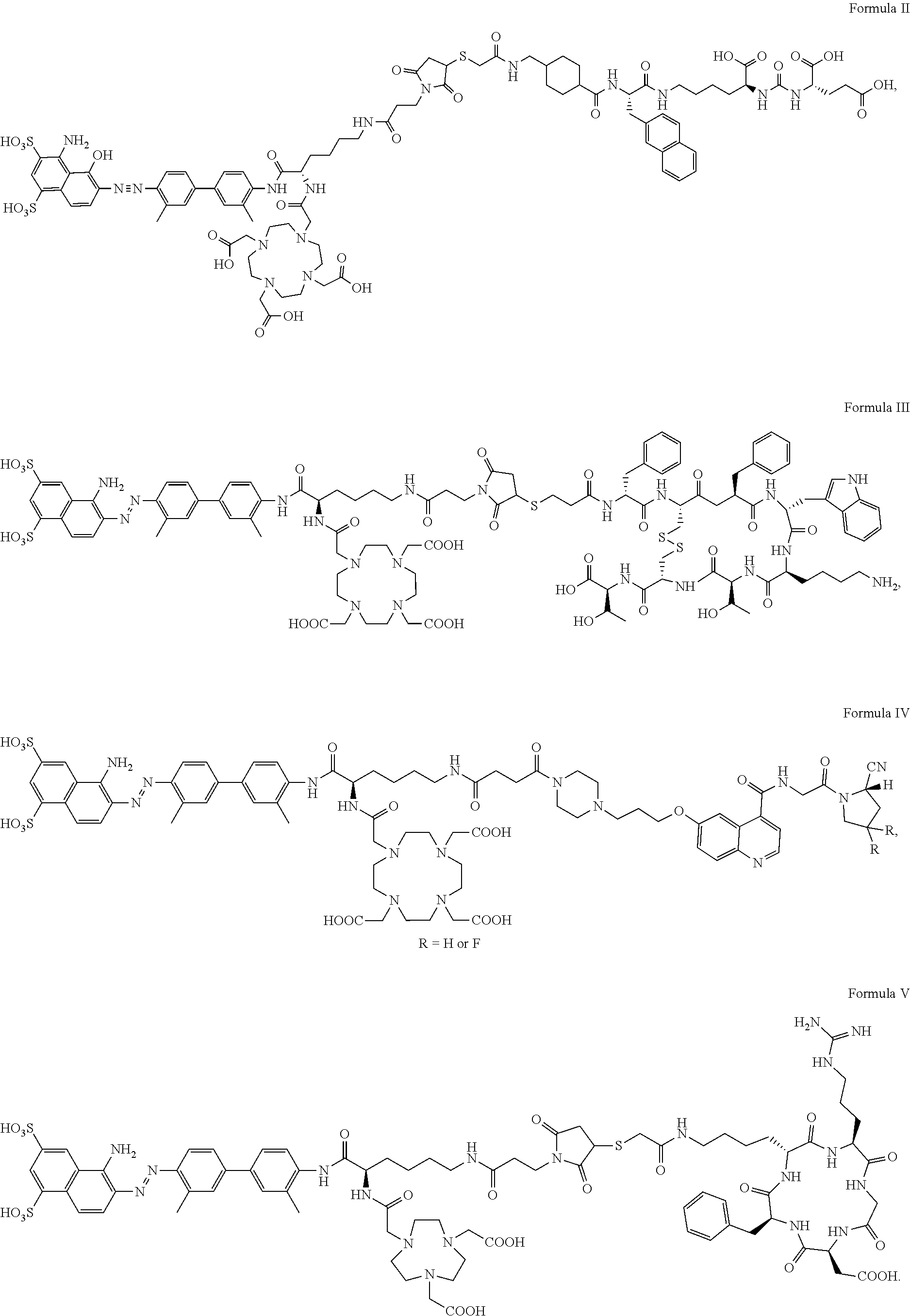

7. The pharmaceutical aqueous solution according to item 1, wherein the radioactive metal nuclide is selected from: ¹⁷⁷Lu, ^{99m}Tc, ⁶⁸Ga, ⁶⁴Cu, ⁶⁷Cu, ¹¹¹In, ⁸⁶Y, ⁹⁰Y, ⁸⁹Zr, ¹⁸⁶Re, ¹⁸⁸Re, ¹⁵³Sm, ⁸²Rb, ¹⁶⁶Ho, ²²⁵Ac, ²¹²Pb, ²¹³Bi ²¹²Bi or ²²⁷Th.
8. The pharmaceutical aqueous solution according to any one of items 1-7, wherein the stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin; preferably one or more selected from the group consisting of: gentisic acid, ethanol and methionine.
9. The pharmaceutical aqueous solution according to item 8, wherein the stabilizer is respectively added during the reaction of forming the complex and after completion of the reaction.
10. The pharmaceutical aqueous solution according to item 9, wherein the stabilizer added during the reaction of forming the complex is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin and melatonin, preferably gentisic acid;
the stabilizer added after completion of the reaction of forming the complex is one or more selected from the group consisting of gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin and melatonin, preferably gentisic acid, ethanol or methionine.
11. The pharmaceutical aqueous solution according to item 10, wherein the stabilizer added during and after the reaction of forming the complex is gentisic acid, and the gentisic acid is present in the pharmaceutical aqueous solution at a total concentration of 0.1-10 mg/mL; preferably 0.5-5 mg/mL.
12. The pharmaceutical aqueous solution according to item 10, wherein the stabilizer added during the reaction of forming the complex is gentisic acid, and the stabilizer added after completion of the reaction of forming the complex is ethanol; gentisic acid is present in the pharmaceutical aqueous solution at a concentration of 0.1-10 mg/mL, preferably 0.5-5 mg/mL; and ethanol is present in the pharmaceutical aqueous solution at a concentration of 0-400 mg/mL.
13. The pharmaceutical aqueous solution according to item 10, wherein the stabilizer added during the reaction of forming the complex is gentisic acid, and the stabilizer added after completion of the reaction of forming the complex is methionine; gentisic acid is present in the pharmaceutical aqueous solution at a concentration of 0.1-10 mg/mL, preferably 0.5-5 mg; and methionine is present in the pharmaceutical aqueous solution at a concentration of 0-50 mg/mL.
14. The pharmaceutical aqueous solution according to item 10, further comprising a buffer solution, wherein the buffer solution may be selected from: acetate, citrate, phosphate or formate solution; and the buffer salt of the buffer solution is present in the pharmaceutical aqueous solution at a concentration of 0.005-0.5M.
15. The pharmaceutical aqueous solution according to item 10, further comprising a cosolvent, wherein the cosolvent is one or more selected from the group consisting of polysorbate 20, polysorbate 40, polysorbate 60, polysorbate 80, poloxamer 188, polyoxyethylene castor oil, Span, ethanol, propylene glycol, glycerol, polyethylene glycol (average molecular weight of 200-8000), sorbitol, dimethyl sulfoxide, and sodium dodecyl sulfate; preferably polysorbate 80.
16. The pharmaceutical aqueous solution according to item 10, further comprising a chelator for free nuclide, wherein the chelator for free nuclide may be selected from pentetic acid and a salt thereof; and the chelator for free nuclide is present in the pharmaceutical aqueous solution at a concentration of 0.005-0.1 mg/mL.
17. The pharmaceutical aqueous solution according to any one of items 1-16, wherein the complex is present in the pharmaceutical aqueous solution at an activity concentration of 0.037-1850 MBq/mL.
18. Use of the pharmaceutical aqueous solution according to any one of items 1-17 in radiotherapy and/or diagnosis of tumors.
19. A method for preparing a radiopharmaceutical aqueous solution, wherein the radiopharmaceutical aqueous solution comprises a complex formed by a radionuclide and an Evans blue derivative molecule, wherein the method comprises the following steps:
mixing a solution comprising a first stabilizer with a solution comprising a radionuclide in a reaction vessel;
after a given time, adding a solution comprising the Evans blue derivative molecule to the reaction vessel, preferably the given time is 0.1-20 min, more preferably 3-10 min; reacting the Evans blue derivative molecule with the radionuclide to obtain the radionuclide complex;
after a given reaction time, adding a solution comprising a second stabilizer to the reaction vessel;
recovering the obtained radiopharmaceutical aqueous solution;
wherein the Evans blue derivative molecule is a compound represented by Formula I or a pharmaceutically acceptable ester, amide, solvate or salt thereof, or a compound represented by Formula I or a salt of a pharmaceutically acceptable ester thereof, or a compound represented by Formula I or a salt of a pharmaceutically acceptable amide thereof, or a compound represented by Formula I or a solvate of a pharmaceutically acceptable ester thereof, or a compound represented by Formula I or a solvate of a pharmaceutically acceptable amide thereof, or a solvate of a compound represented by Formula I or a pharmaceutically acceptable salt thereof,

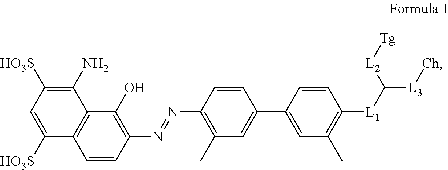

wherein
L₁ is —(CH₂)ₘ—, wherein m is an integer from 0 to 12, wherein each CH₂ may be individually replaced with —O—, —NH(CO)— or —(CO)NH—, providing that no two adjacent CH₂ groups are replaced;
L₂ is a C₁-C₆₀ linking group, optionally including —O—, —S—, —S(O)—, —S(O)₂—, —N(R)—, —C(═O)—, —C(═O)O—, —OC(═O)—, —N(R)C(═O)—, —C(═O)N(R)—, —OC(═O)O—, —N(R)C(═O)O—, —OC(═O)N(R)—,

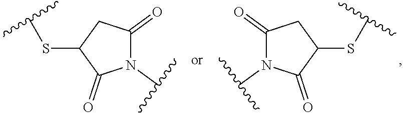

wherein each R is H or C₁-C₆ alkyl;
L₃ is —(CH₂)ₙ—, wherein n is an integer from 0 to 12, wherein each CH₂ may be individually replaced with —O—, —NH(CO)— or —(CO)NH—, providing that no two adjacent CH₂ groups are replaced;
Ch is a chelating group; and
Tg is a targeting group.
20. The method according to item 19, wherein the radionuclide is selected from: ¹⁷⁷Lu, ^{99m}Tc, ⁶⁸Ga, ⁶⁴Cu, ⁶⁷Cu, ¹¹¹In, ⁸⁶Y, ⁹⁰Y, ⁸⁹Zr, ¹⁸⁶Re, ¹⁸⁸Re, ¹⁵³Sm, ⁸²Rb, ¹⁶⁶Ho, ²²⁵Ac, ²¹²Pb, ²¹³Bi, ²¹²Bi or ²²⁷Th.
21. The method according to item 19, wherein Ch in Formula I is selected from:

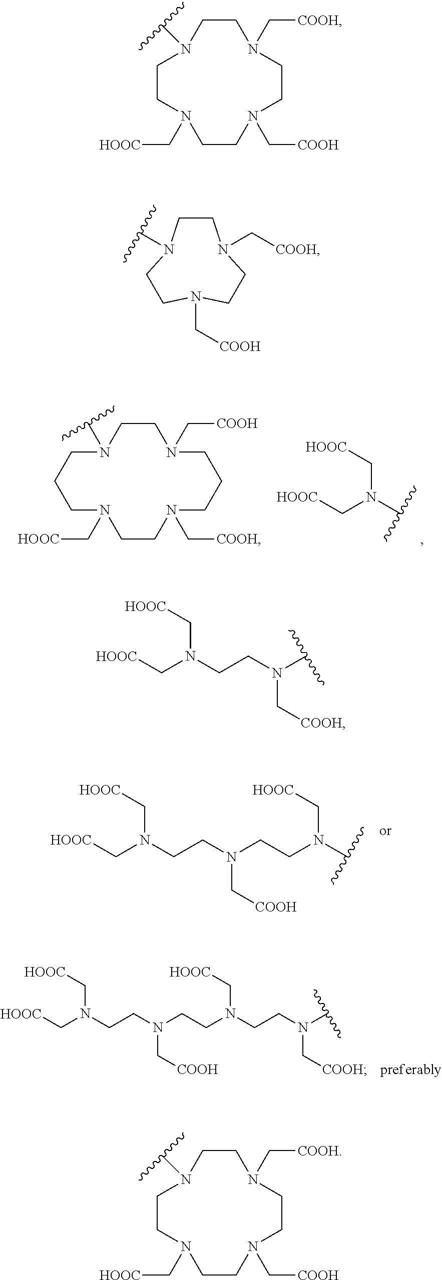

22. The method according to item 19, wherein Tg in Formula I is selected from a chemical group capable of targeting somatostatin receptor (SSTR), prostate-specific membrane antigen (PSMA), fibroblast activation protein (FAP), folate receptor (FR), epidermal growth factor receptor or integrin.
23. The method according to item 19, wherein the Evans blue derivative molecule is selected from the compounds represented by Formula II, Formula III, Formula IV or Formula V

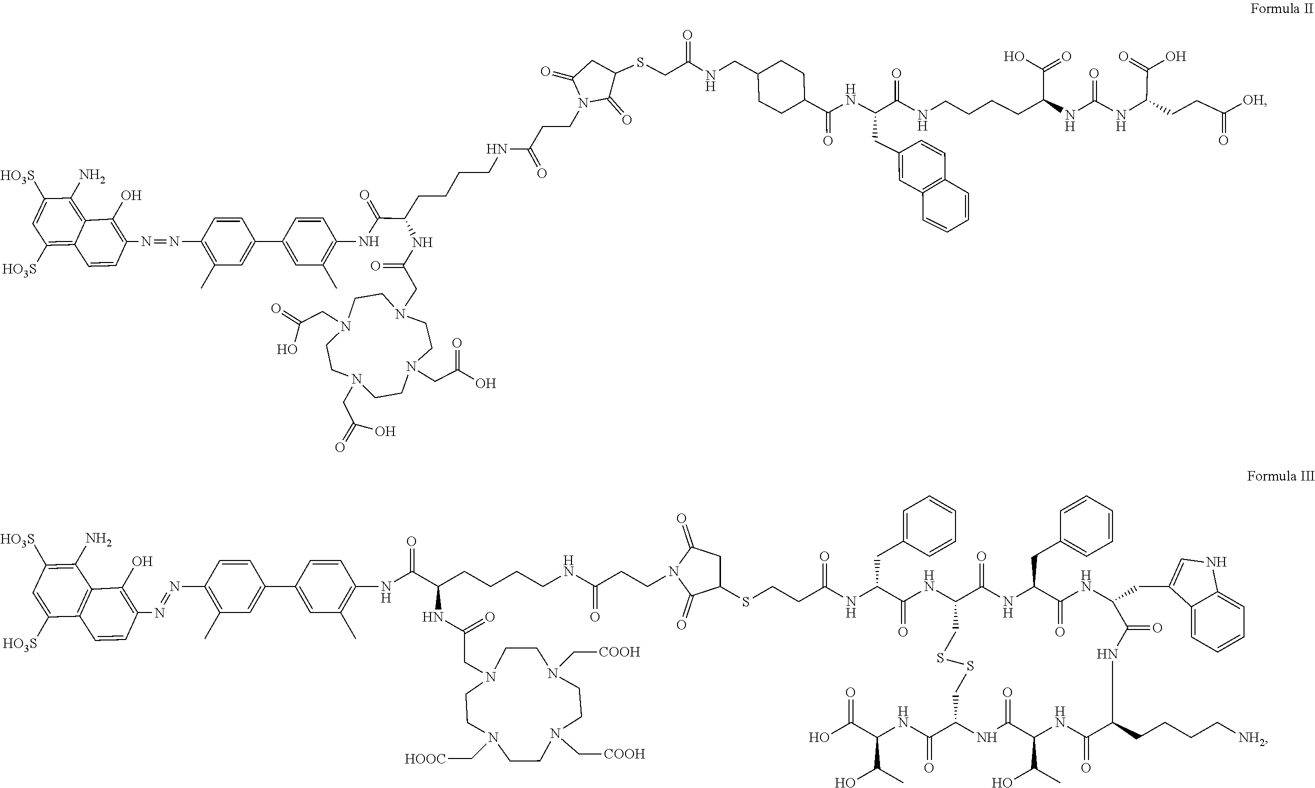

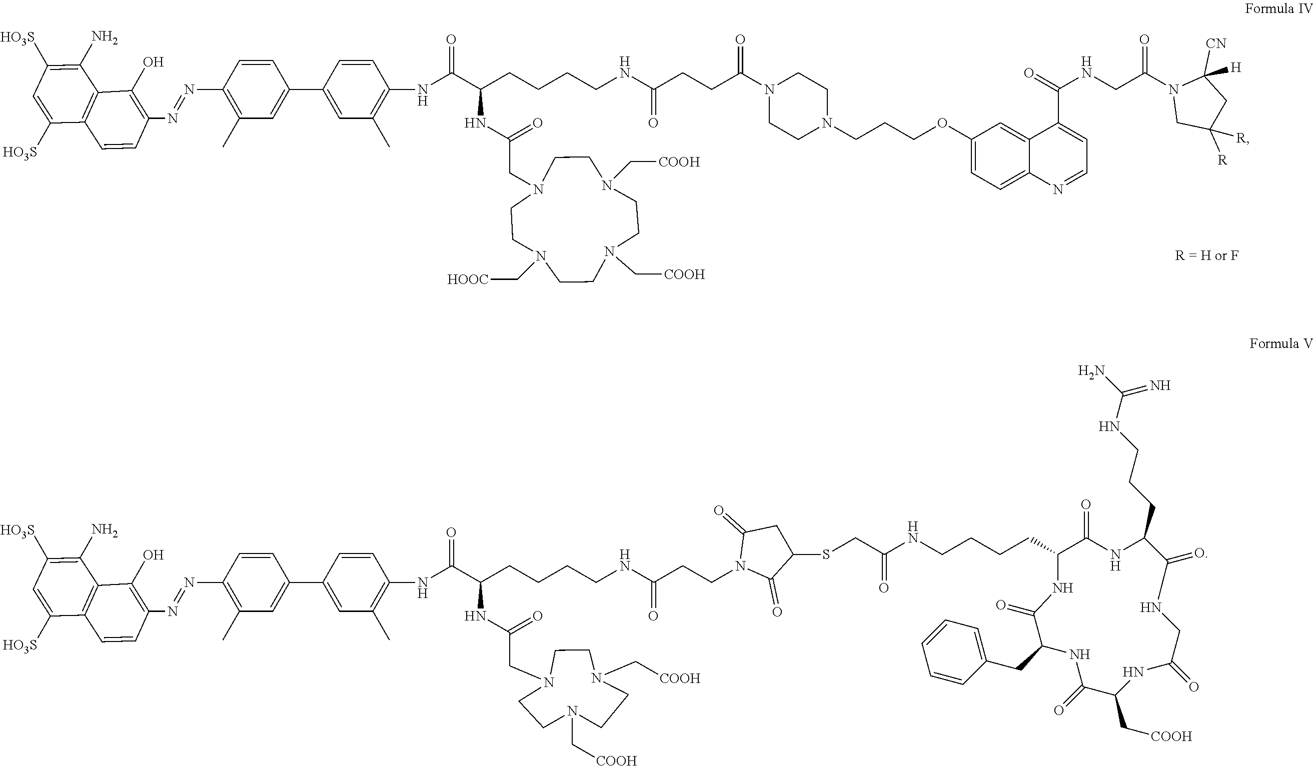

24. The method according to any one of items 19-23, wherein the solution comprising the radionuclide is taken out from the raw material bottle and then added to the reaction vessel, and the method further comprises:
rinsing the raw material bottle with a rinse solution, and the rinsed solution is transferred into the reaction vessel to be mixed with the solution comprising the radionuclide.
25. The method according to item 24, wherein the rinse solution is an aqueous solution, preferably selected from: a solution comprising a first stabilizer, a solution comprising a buffered salt, water or sodium chloride injection; more preferably rinsing with the rinse solution is repeated one or more times.
26. The method according to item 19, wherein in the step of reacting the Evans blue derivative molecule with the radionuclide, the molar ratio of the Evans blue derivative molecule to the radionuclide is 1.5-50, preferably 5-20.
27. The method according to item 19, wherein in the step of reacting the Evans blue derivative molecule with the radionuclide, the reaction temperature is 50-100° C., preferably 60-80° C.; and the reaction time is 5-60 min, preferably 10-30 min.
28. The method according to item 19, wherein the first stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin and melatonin, preferably gentisic acid.
29. The method according to item 28, wherein in the step of reacting the Evans blue derivative molecule with the radionuclide, the concentration of the first stabilizer in the reaction phase solution is 0.6-20.0 mg/mL.
30. The method according to item 19, wherein the second stabilizer is one or more selected from the group consisting of gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin and melatonin; preferably gentisic acid, ethanol or methionine.
31. The method according to item 30, wherein in the radiopharmaceutical aqueous solution, the concentration of the second stabilizer is 0-400 mg/mL.
32. The method according to item 19, wherein a buffer salt solution is added before reacting the Evans blue derivative molecule with the radionuclide, preferably the buffer salt solution is present in the solution comprising the first stabilizer.
33. The method according to item 32, wherein the buffer salt solution is selected from: acetate, citrate, phosphate or formate solution, preferably an acetic acid-sodium acetate buffer salt solution.
34. The method according to item 19, wherein the step of adding a solution comprising a second stabilizer to the reaction vessel after a given reaction time further comprises adding a cosolvent to the reaction vessel.
35. The method according to item 34, wherein the cosolvent is one or more selected from the group consisting of: polysorbate 20, polysorbate 40, polysorbate 60, polysorbate 80, poloxamer 188, polyoxyethylene castor oil, Span, ethanol, propylene glycol, glycerol, polyethylene glycol (average molecular weight of 200-8000), sorbitol, dimethyl sulfoxide, and sodium dodecyl sulfate, preferably polysorbate 80.
36. The method according to item 19, wherein the step of adding a solution comprising a second stabilizer to the reaction vessel after a given reaction time further comprises adding a chelator for free nuclide to the reaction vessel, and the chelator may be selected from pentetic acid and a salt thereof, preferably pentetic acid.
37. The method according to any one of items 19-36, wherein it further comprises filtering the radiopharmaceutical aqueous solution through a 0.22 μm filter membrane for sterilization, preferably the filtration sterilization is performed after adding the solution comprising the second stabilizer.
38. The method according to any one of items 19-37, wherein it further comprises diluting the radioactive aqueous solution, preferably sodium chloride injection is added for dilution after adding the solution comprising the second stabilizer.
39. A radiopharmaceutical aqueous solution prepared by the method according to any one of items 19-38.
40. The radiopharmaceutical aqueous solution according to item 39, wherein the radiopharmaceutical aqueous solution is the radiopharmaceutical aqueous solution according to any one of items 1-17.
41. Use of the method according to any one of items 19-38 in radiotherapy and/or diagnosis of tumors.
42. A radiotherapy method for tumors, wherein it comprises administering the pharmaceutical aqueous solution according to any one of items 1-17 to a subject in need thereof.

### Effects of the Application

The radiopharmaceutical aqueous solution provided by the present application has the following beneficial effects:

In one embodiment, the stabilizer may be a single type, or a combination of two or more types. In some embodiments, it is preferred to use only gentisic acid, or gentisic acid and ethanol, as ethanol has a good anti-radiolysis effect. At the same time, it is preferred not to use ascorbic acid as the stabilizer. The addition of ascorbic acid is not conducive to the stability of Evans blue derivatives.

In one embodiment, pentetic acid is added as a chelator after the reaction is completed, so as to chelate unreacted free radionuclide ions to reduce unnecessary irradiation of healthy tissues by free radionuclide ions in the body.

In one embodiment, the pharmaceutical aqueous solution of the present application has an initial radiochemical purity of not less than 93%, preferably not less than 95%, and more preferably not less than 97% after the radiopharmaceutical is prepared. In one embodiment, the radiochemical purity of the API of the pharmaceutical aqueous solution of the Evans blue derivative according to the present application is maintained at not less than 90% within 48 hours, preferably not less than 90% within 72 hours, under storage conditions of 32° C. and 60% RH.

The method for preparing a radiopharmaceutical aqueous solution provided by the present application has the following beneficial effects:

In the present application, the concentration of gentisic acid in the reaction phase solution is controlled to be 0.6-20.0 mg/mL. When the concentration is lower than 0.6 mg/mL, the anti-radiolysis effect of gentisic acid is insufficient; and when the concentration is higher than 20.0 mg/mL, the high concentration of gentisic acid will retard the reaction kinetics, and unfavorably prolong the time required for the reaction. This control range is to minimize the concentration of gentisic acid to avoid adverse effects on reaction kinetics while ensuring stability of the solution.

Before the solution comprising the Evans blue derivative molecules is mixed with the nuclide solution, firstly the nuclide solution is mixed with a solution comprising a first stabilizer, and after a given time, a solution comprising the Evans blue derivative molecules is added, so as to allow the first stabilizer to fully contact with the nuclide solution and quench a large number of free radicals caused by radiolysis in the nuclide solution, thereby protecting the Evans blue derivative molecules added to the reaction system later from being attacked by active free radicals, and ensuring the initial radiochemical purity of the product. The process can achieve an initial radiochemical purity of 95.0-99.5%, while the initial radiochemical purity of a product obtained by the synthesis process of directly mixing a solution comprising the Evans blue derivative molecules with the nuclide solution is about 89%-93%.

The process method according to the present application can at least ensure that the marking rate is above 90%, preferably above 95%, and most preferably above e 99%. In some embodiments provided in the present application, there is no purification step for removing radioactive impurities after the reaction, such as preparative liquid phase separation, solid phase extraction separation, etc.

If the radionuclide complex solution is required to be sterile, the step of recovering the product further comprises passing the obtained solution through a 0.22 μm sterilizing filter membrane, and the solution may be further diluted according to the dosage.

### DETAILED DESCRIPTION

The present application is further described in detail below in conjunction with particular embodiments. The examples given are intended to enable a more thorough understanding of the present application, and to fully convey the scope of the present application to those skilled in the art.

It should be noted that, certain terms are used in the specification and claims to refer to specific components. It should be understood by those skilled in the art that, the technicians may use different terms to refer to the same component. This specification and claims do not use differences in nouns as a way to distinguish components, but use differences in functions of components as the criterion for distinction. As mentioned throughout the specification and claims, “comprise/comprising” or “include/including” are open-ended terms, and should be interpreted as “including but not limited to”. Hereinafter, the preferred embodiments of the present application are described, however, the description is for the purpose of the general principles of the specification, and is not intended to limit the scope of the present application. The protection scope of this application shall be determined by the appended claims.

The present application relates to a radiopharmaceutical aqueous solution, which comprises: a complex formed by a targeting molecule modified with an Evans blue (EB) fragment and a radioactive metal nuclide, and a stabilizer.

In a particular embodiment, the targeting molecule modified with the Evans blue (EB) fragment (or the “Evans blue derivative molecule” described in the specification of this application) is a compound represented by Formula I or a pharmaceutically acceptable ester, amide, solvate, salt thereof, or a salt of a compound represented by Formula I or a pharmaceutically acceptable ester thereof, or a salt of a compound represented by Formula I or a pharmaceutically acceptable amide thereof, or a solvate of a compound represented by Formula I or a pharmaceutically acceptable ester thereof, or a solvate of a compound represented by Formula I or a pharmaceutically acceptable amide thereof, or a solvate of a compound represented by Formula I or a pharmaceutically acceptable salt thereof;

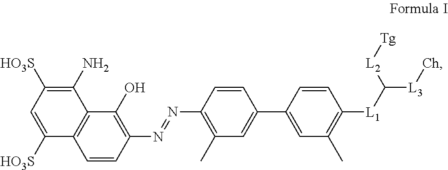

wherein
L₁ is —(CH₂)ₘ—, wherein m is an integer from 0 to 12, wherein each CH₂ may be individually replaced with —O—, —NH(CO)— or —(CO)NH—, providing no two adjacent CH₂ groups are replaced;
L₂ is a C₁-C₆₀ linking group, optionally including —O—, —S—, —S(O)—, —S(O)₂—, —N(R)—, —C(═O)—, —C(═O)O—, —OC(═O)—, —N(R)C(═O)—, —C(═O)N(R)—, —OC(═O)O—, —N(R)C(═O)O—, —OC(═O)N(R)—,

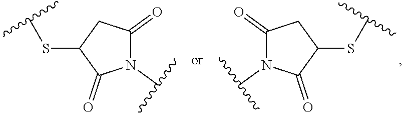

wherein each R is H or C₁-C₆ alkyl;
L₃ is —(CH₂)ₙ—, wherein n is an integer from 0 to 12, wherein each CH₂ may be individually replaced with —O—, —NH(CO)— or —(CO)NH—, providing no two adjacent CH₂ groups are replaced;
Ch is a chelating group; and
Tg is a targeting group.

Evans Blue (EB) is a non-membrane permeable azo dye preparation. Because of its high affinity to serum albumin in the blood, its reversible binding property with albumin is used to modify a targeting molecule with a truncated EB fragment (tEB), so that the targeting molecule can reversibly bind to endogenous serum albumin via the tEB fragment, and serum albumin is used as a reversible carrier of a drug molecule to prolong the half-life of the drug molecule in the blood, thereby increasing the availability of the drug molecule, and further increasing the accumulation and retention time of the drug molecule in the tumor. The structural formula of Evans Blue (EB) dye is represented by the following Formula VI:

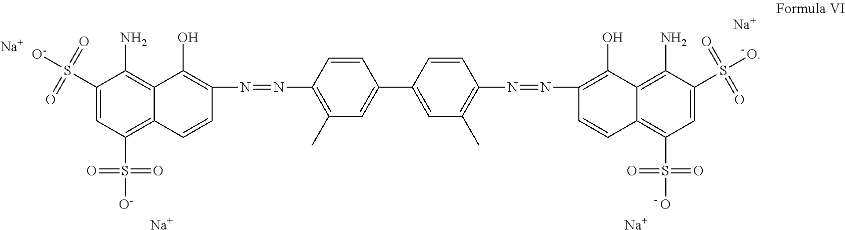

In some particular embodiments of the present application, the Evans blue derivative molecule is a compound of Formula I. In other particular embodiments, the Evans blue derivative molecule is a pharmaceutically acceptable ester, amide, solvate or salt of the compound of Formula I. In other particular embodiments, the Evans blue derivative molecule is a salt of a pharmaceutically acceptable ester of the compound of Formula I. In other particular embodiments, the Evans blue derivative molecule is a salt of a pharmaceutically acceptable amide of the compound of Formula I. In other particular embodiments, the Evans blue derivative molecule is a solvate of a pharmaceutically acceptable ester of the compound of Formula I. In other particular embodiments, the Evans blue derivative molecule is a solvate of a pharmaceutically acceptable amide of the compound of Formula I. In other particular embodiments, the Evans blue derivative molecule is a solvate of a pharmaceutically acceptable salt of the compound of Formula I. The Evans blue derivative molecules may be synthesized from parent compounds comprising a basic or acidic moiety by conventional chemical methods.

In a particular embodiment, L₁ in Formula I is —NH(CO)—, and L₃ is —NH(CO)CH₂—, that is, the compound of Formula I is a compound represented by the following Formula VII:

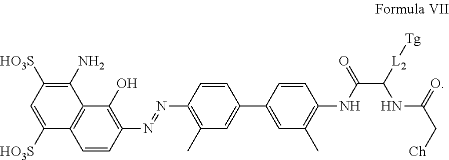

In a particular embodiment, the chelating group Ch in Formula I is selected from:

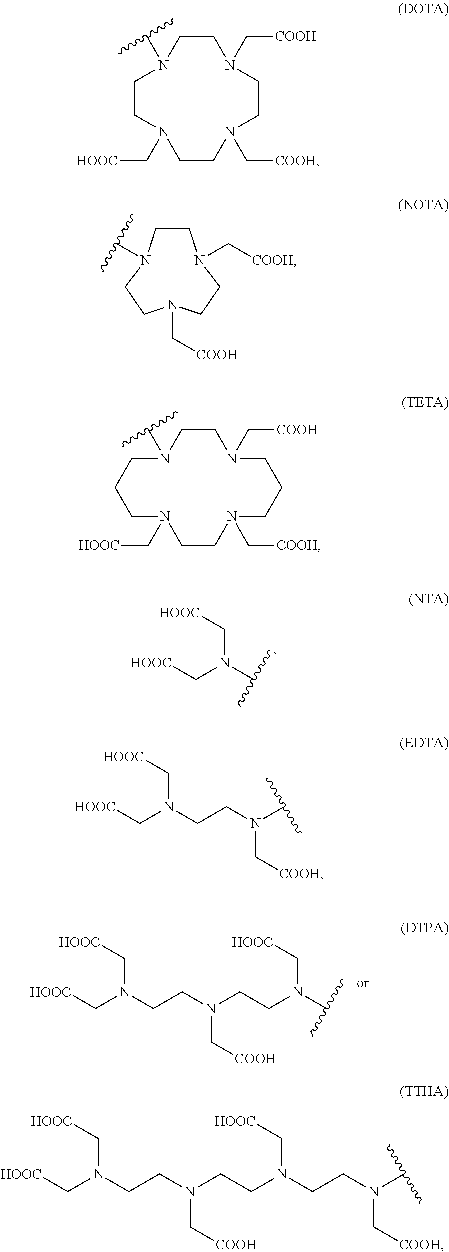

preferably the chelating group Ch in Formula I is

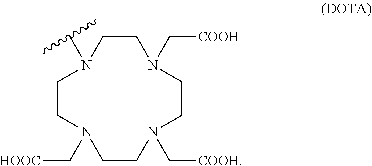 [SMILES: *N1CCN(CC(=O)O)CCN(CC(=O)O)CCN(CC(=O)O)CC1]

A chelating group is a group having two or more coordinating atoms and capable of combining with the same central atom to form a ring structure, which can form two or more separate coordination bonds with a single central atom, usually a metal ion. The chelating group in the present application is an organic group having a plurality of N, O or S heteroatoms and having a structure that allows two or more heteroatoms to form bonds with the same metal ion. In a particular embodiment of the present application, the chelating group is used to form a bond structure with a radioactive metal nuclide.

In a particular embodiment of the present application, the targeting group Tg in Formula I is a chemical group specifically targeting a certain biological target. In some embodiments, Tg is a chemical group capable of targeting a biological target selected from: somatostatin receptor (SSTR), prostate specific membrane antigen (PSMA), fibroblast activation protein (FAP), folate receptor (FR), epidermal growth factor receptor or integrin. In some particular embodiments, the targeting group Tg is selected from:

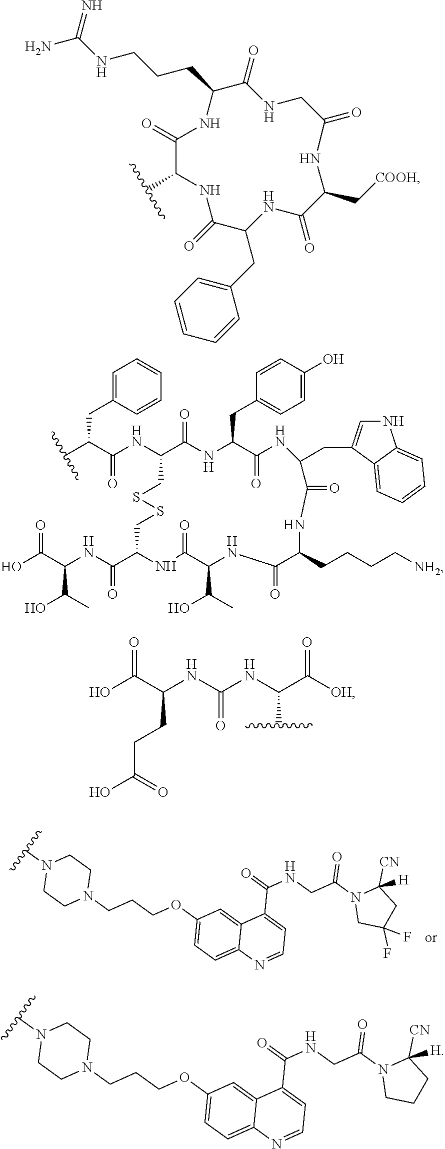

In a particular embodiment of the present application, the compound of Formula I is EB-PSMA, and the structural formula is represented by the following Formula II:

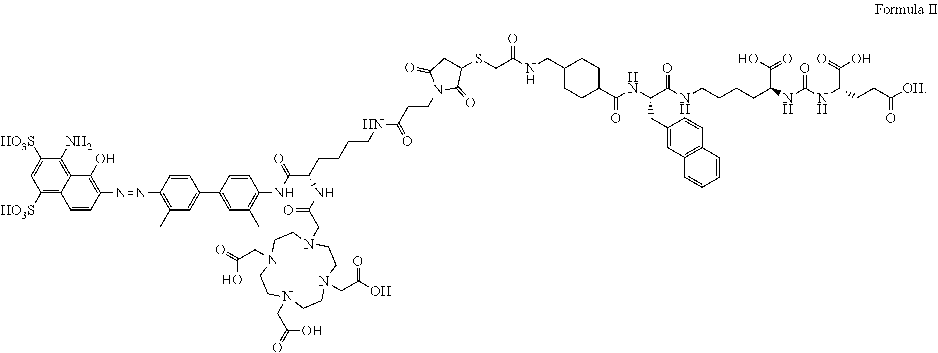 [SMILES: Cc1cc(-c2ccc(NC(=O)[C@H](CCCCNC(=O)CCN3C(=O)CC(SCC(=O)NCC4CCC(C(=O)N[C@@H](Cc5ccc6ccccc6c5)C(=O)NCCCC[C@H](NC(=O)N[C@@H](CCC(=O)O)C(=O)O)C(=O)O)CC4)C3=O)NC(=O)CN3CCN(CC(=O)O)CCN(CC(=O)O)CCN(CC(=O)O)CC3)c(C)c2)ccc1N=Nc1ccc2c(S(=O)(=O)O)cc(S(=O)(=O)O)c(N)c2c1O]

In a particular embodiment of the present application, the compound of Formula I is DOTA-EB-TATE, and the structural formula is represented by the following Formula III:

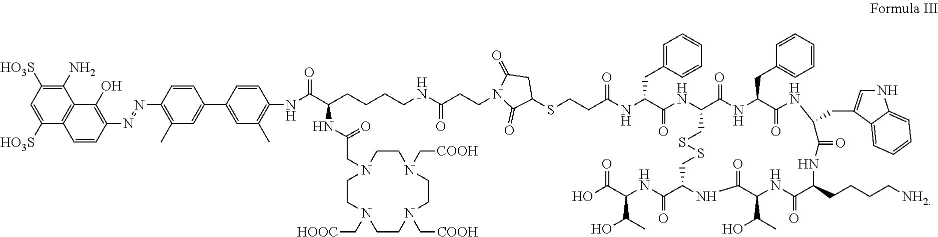 [SMILES: Cc1cc(-c2ccc(NC(=O)[C@@H](CCCCNC(=O)CCN3C(=O)CC(SCCC(=O)N[C@H](Cc4ccccc4)C(=O)N[C@H]4CSSC[C@@H](C(=O)N[C@H](C(=O)O)C(C)O)NC(=O)[C@H](C(C)O)NC(=O)[C@H](CCCCN)NC(=O)[C@@H](Cc5c[nH]c6ccccc56)NC(=O)[C@H](Cc5ccccc5)NC4=O)C3=O)NC(=O)CN3CCN(CC(=O)O)CCN(CC(=O)O)CCN(CC(=O)O)CC3)c(C)c2)ccc1/N=N/c1ccc2c(S(=O)(=O)O)cc(S(=O)(=O)O)c(N)c2c1O]

In a particular embodiment of the present application, the compound of Formula I is EB-FAPI, and the structural formula is represented by the following Formula IV:

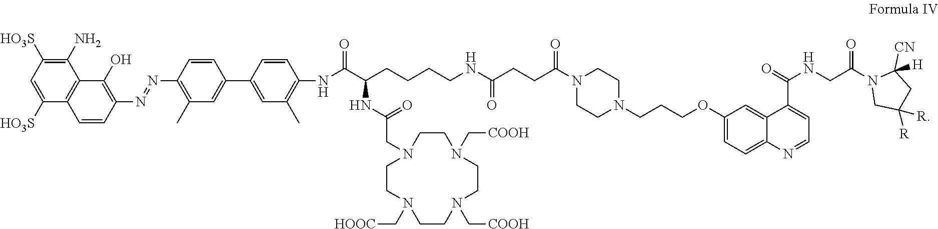

In a particular embodiment of the present application, the compound of Formula I is NMEB-RGD, and the structural formula is represented by the following Formula V:

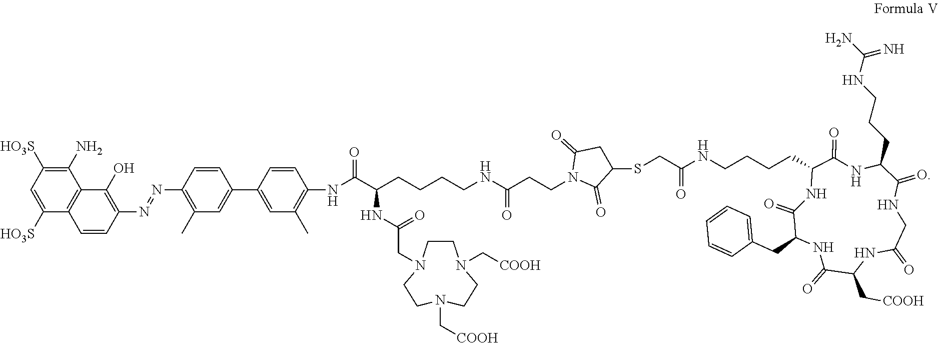 [SMILES: Cc1cc(-c2ccc(NC(=O)[C@@H](CCCCNC(=O)CCN3C(=O)CC(SCC(=O)NCCCC[C@H]4NC(=O)C[C@H](Cc5ccccc5)NC(=O)[C@H](CC(=O)O)NC(=O)CNC(=O)[C@H](CCCNC(=N)N)NC4=O)C3=O)NC(=O)CN3CCN(CC(=O)O)CCN(CC(=O)O)CC3)c(C)c2)ccc1/N=N/c1ccc2c(S(=O)(=O)O)cc(S(=O)(=O)O)c(N)c2c1O]

In a particular embodiment of the present application, the radioactive metal nuclide that forms a complex with the Evans blue derivative molecule is selected from: ¹⁷⁷Lu, ^{99m}Tc, ⁶⁸Ga, ⁶⁴Cu, ⁶⁷Cu, ¹¹¹In, ⁸⁶Y, ⁹⁰Y, ⁸⁹Zr, ¹⁸⁶Re, ¹⁸⁸Re, ¹⁵³Sm, ⁸²Rb, ¹⁶⁶Ho, ²²⁵Ac, ²¹²Pb, ²¹³Bi, ²¹²Bi or ²²⁷Th. The radioactive metal nuclide may be bound to the chelating group Ch by chelation, or by other means, such as conventional covalent or ionic bonds known in the chemical art. The radionuclide may be of appropriate purpose, such as radiotherapy and/or diagnosis.

In a particular embodiment of the present application, the radioactive metal nuclide is present in the pharmaceutical aqueous solution preparation at a volume radioactivity concentration of 0.037-1850 MBq/mL.

In a particular embodiment of the present application, the stabilizer in the aqueous solution of the radioactive drug is an anti-radioactive decomposition and degradation stabilizer. Particularly, the stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin, preferably one or more selected from the group consisting of gentisic acid, ethanol and methionine.

In a particular embodiment, the total concentration of the stabilizer in the pharmaceutical aqueous solution is 0.5-400 mg/mL, for example, 0.5, 1, 2, 5, 10, 25, 50, 100, 150, 200, 250, 300, 350, or 400 mg/mL. Preferably, it is 1-80 mg/mL, for example, it may be 1, 2, 5, 10, 15, 20, 25, 30, 35, 40, 45, 50, 55, 60, 65, 70, 75, or 80 mg/mL.

In a particular embodiment, the stabilizer is respectively added during the complex reaction of forming the nuclide complex and after completion of the reaction. Particularly, “adding during the complex reaction” means that the stabilizer, and the radionuclide solution and the Evans blue derivative molecule solution for forming the complex together form a reaction phase solution when conditions sufficient for the complex reaction to occur are met; and “adding after completion of the reaction” means that the stabilizer is added after the complex reaction has occurred for a certain period of time and the complex has been formed. Furthermore, the stabilizer added during the complex reaction is a first stabilizer, and the stabilizer added after completion of the reaction is a second stabilizer. The first stabilizer is generally a small molecule compound with antioxidant properties, which is used to reduce radiolysis under high radiation. The main function of the secondary stabilizer is to maintain the stability of the formulation during storage. The first stabilizer and the second stabilizer may be selected from the same stabilizer or different stabilizers.

In a particular embodiment, the first stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin, preferably gentisic acid.

In a particular embodiment, the second stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin, preferably gentisic acid, ethanol or methionine.

In a preferred embodiment, the first stabilizer and the second stabilizer are the same, and are both selected from gentisic acid or a salt thereof. Particularly, gentisic acid (i.e., the first stabilizer) is added during the complex reaction, and its concentration range in the reaction system is 0.6-20 mg/mL, preferably 2-10 mg/mL, for example, it may be 2, 2.5, 3, 3.5, 4, 4.5, 5, 5.5, 6, 6.5, 7, 7.5, 8, 8.5, 9, 9.5, or 10 mg/mL. After the reaction is completed, gentisic acid (i.e., the second stabilizer) is continued to be added to the preparation to make gentisic acid exist in the entire pharmaceutical aqueous solution at a total concentration of 0.1-10 mg/mL, preferably 0.5-5 mg/mL, for example, 0.5, 0.8, 1.0, 1.2, 1.5, 2.0, 2.3, 2.4, 2.5, 2.6, 2.7, 2.8, 3.0, 3.2, 3.5, 3.8, 4.0, 4.5, or 5.0 mg/mL.

In other preferred embodiments, the stabilizer is two different stabilizers.

In a particular embodiment, the first stabilizer added to the reaction system during the complex reaction is gentisic acid or a salt thereof, which is present in the pharmaceutical aqueous solution at a concentration of 0.5-5 mg/mL, preferably 0.5-2 mg/mL, for example, 0.5, 0.8, 1.0, 1.2, 1.5, 1.8, or 2.0 mg/mL. The second stabilizer added after completion of the reaction is ethanol, which is present in the pharmaceutical aqueous solution at a concentration of 0-400 mg/mL, preferably 10-120 mg/mL, for example, 10, 30, 50, 60, 70, 80, 100, or 120 mg/mL, and its volume fraction is 0%-50%, preferably 1%-15%, for example, 1%, 3%, 5%, 6%, 7%, 8%, 9%, 10%, 12%, or 15%.

In a particular embodiment, the first stabilizer added to the reaction system during the complex reaction is gentisic acid or a salt thereof, which is present in the pharmaceutical aqueous solution at a concentration of 0.5-5 mg/mL, preferably 0.5-2 mg/mL, for example, 0.5, 0.8, 1.0, 1.2, 1.3, 1.4, 1.5, 1.6, 1.8, or 2.0 mg/mL. The second stabilizer added after completion of the reaction is L-methionine, which is present in the pharmaceutical aqueous solution at a concentration of 0-50 mg/mL, preferably 1-10 mg/mL, for example, 1, 2, 3, 4, 5, 6, 7, 8, 9, or 10 mg/mL.

In other particular embodiments, the two stabilizers preferably do not comprise ascorbic acid and a salt thereof.

In a particular embodiment, the pharmaceutical aqueous solution also comprises a buffer solution. The buffer solution may be added during the complex reaction to adjust the pH of the reaction phase solution, and it can also be added again after completion of the reaction to adjust the pH of the preparation solution. The two buffer solutions added twice may be the same or different. The buffer solution may be a solution of an acetate system (such as acetic acid-sodium acetate system, or sodium acetate system), a citrate system (such as citric acid-sodium citrate system), a phosphate system (such as sodium dihydrogen phosphate-disodium hydrogen phosphate system), or a formate system (such as formic acid-sodium formate system). In a preferred embodiment, the concentration of the buffer salt in the reaction phase solution is 0.01-2.0M. In a preferred embodiment, the total buffer salt concentration in the final pharmaceutical aqueous solution is 0.005-0.5M.

In a particular embodiment, the pharmaceutical aqueous solution also comprises a cosolvent, which functions to reduce the adsorption of the API on the surfaces of various contact materials (especially glass and plastic surfaces). The cosolvent is one or more selected from the group consisting of: polysorbate 20, polysorbate 40, polysorbate 60, polysorbate 80, poloxamer 188, polyoxyethylene castor oil, span, ethanol, propylene glycol, glycerol, polyethylene glycol (average molecular weight of 200-8000), sorbitol, dimethyl sulfoxide, and sodium dodecyl sulfate, preferably polysorbate 80. In a particular embodiment, the concentration of the cosolvent in the pharmaceutical aqueous solution is 0.01-10 mg/mL, preferably 0.05-1.0 mg/mL, for example, 0.05, 0.08, 0.1, 0.2, 0.3, 0.4, 0.5, 0.6, 0.7, 0.8, or 1.0 mg/mL.

In a particular embodiment, the pharmaceutical aqueous solution also comprises a chelator for free metal nuclides. The chelator functions to have a complex reaction with the unreacted free nuclide ions in the pharmaceutical aqueous solution, so as to reduce unnecessary irradiation of the free radionuclide ions to healthy tissues in the body. Therefore, the chelator is required to have a strong ability to have a complex reaction with radionuclide ions. Even after the injection solution enters the body and is diluted by plasma, it can still react rapidly with free radionuclide ions under lower concentration conditions. This complex reaction needs to be rapid under mild conditions, and it may be performed completely at room temperature. In a particular embodiment, the chelator is pentetic acid or a salt thereof, preferably pentetic acid. The concentration of the chelator in the pharmaceutical aqueous solution is 0.005-0.1 mg/mL, for example, 0.005, 0.01, 0.02, 0.03, 0.04, 0.05, 0.06, 0.07, 0.08, 0.09, or 0.1 mg/mL. Within this range, pentetic acid has sufficient chelating ability for free nuclide ions, and the complex can maintain stability for at least 48 hours under radiolysis, and preferably can maintain stability for 72 hours, that is, the free nuclide ions will not be released due to the radiolysis of the chelator.

The present application also provides a technical solution for applying the pharmaceutical aqueous solution to radiotherapy and/or diagnosis of tumors, comprising administering to a patient an effective amount of the pharmaceutical aqueous solution, or a composition consisting of the pharmaceutical aqueous solution and one or more other tumor immunological therapeutic agents. In some particular embodiments, the pharmaceutical aqueous solution or a composition comprising the same may be used to treat a neuroendocrine tumor, prostate cancer, breast cancer, ovarian cancer, pancreatic cancer, liver cancer, lung cancer, colorectal cancer, or melanoma, etc. In other particular embodiments, the pharmaceutical aqueous solution or a composition comprising the same provided in the present application can also be used to prepare a medicament for preventing or treating a diabetes and Alzheimer's disease.

In a particular embodiment, the pharmaceutical aqueous solution provided by the present application can at least ensure that the radiochemical purity of the API is not less than 90% within 48 hours, more preferably not less than 90% within 72 hours, under storage conditions of 32° C. and 60% RH, wherein the radiochemical purity is a value determined by HPLC.

The present application also relates to a method for preparing a radiopharmaceutical aqueous solution, in a particular embodiment, the method comprises the following steps:
mixing a solution comprising a first stabilizer with a solution comprising a radionuclide in a reaction vessel;
after a given time, adding a solution comprising the Evans blue derivative molecule to the reaction vessel, preferably the given time is 0.1-20 min, more preferably 3-10 min;
reacting the Evans blue derivative molecule with the radionuclide to obtain the radionuclide complex;
after a given reaction time, adding a solution comprising a second stabilizer to the reaction vessel;
recovering the obtained radiopharmaceutical aqueous solution;
wherein the Evans blue derivative molecule is a compound represented by Formula I or a pharmaceutically acceptable ester, amide, solvate or salt thereof, or a compound represented by Formula I or a salt of a pharmaceutically acceptable ester thereof, or a compound represented by Formula I or a salt of a pharmaceutically acceptable amide thereof, or a compound represented by Formula I or a solvate of a pharmaceutically acceptable ester thereof, or a compound represented by Formula I or a solvate of a pharmaceutically acceptable amide thereof, or a solvate of a compound represented by Formula I or a pharmaceutically acceptable salt thereof,

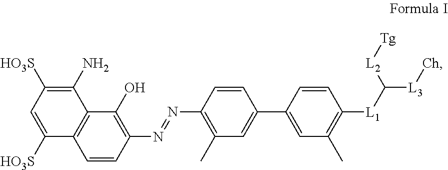

wherein
L₁ is —(CH₂)ₘ—, wherein m is an integer from 0 to 12, wherein each CH₂ may be individually replaced with —O—, —NH(CO)— or —(CO)NH—, providing no two adjacent CH₂ groups are replaced;
L₂ is a C₁-C₆₀ linking group, optionally including —O—, —S—, —S(O)—, —S(O)₂—, —N(R)—, —C(═O)—, —C(═O)O—, —OC(═O)—, —N(R)C(═O)—, —C(═O)N(R)—, —OC(═O)O—, —N(R)C(═O)O—, —OC(═O)N(R)—,

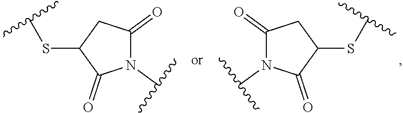

wherein each R is H or C₁-C₆ alkyl;
L₃ is —(CH₂)ₙ—, wherein n is an integer from 0 to 12, wherein each CH₂ may be individually replaced with —O—, —NH(CO)— or —(CO)NH—, providing no two adjacent CH₂ groups are replaced;
Ch is a chelating group; and
Tg is a targeting group.

In one embodiment of the present application, the radiopharmaceutical aqueous solution comprises a radionuclide complex, and the radionuclide complex is formed by a radionuclide and the Evans blue derivative molecule.

In a particular embodiment of the present application, the solution comprising radionuclides is a solution comprising radioactive metal elements. In some particular embodiments, the radionuclide is selected from: ¹⁷⁷Lu, ^{99m}Tc, ⁶⁸Ga, ⁶⁴Cu, ⁶⁷Cu, ¹¹¹In, ⁸⁶Y, ⁹⁰Y, ⁸⁹Zr, ¹⁸⁶Re, ¹⁸⁸Re, ¹⁵³Sm, ⁸²Rb, ¹⁶⁶Ho, ²²⁵Ac, ²¹²Pb, ²¹³Bi, ²¹²Bi or ²²⁷Th. In a particular embodiment, the radionuclide is ¹⁷⁷Lu, and in the step of having a complex reaction with the Evans blue derivative molecule, the specific activity of the radionuclide is not less than 20 Ci/mg, preferably not less than 60 Ci/mg, and most preferably not less than 80 Ci/mg. A radionuclide with too low specific activity will affect the radiolabeling efficiency.

In the present application, the Evans Blue derivative molecule is a targeting molecule modified with a truncated Evans Blue fragment (tEB). In some particular embodiments, the Evans blue derivative molecule is a compound of Formula I. In other particular embodiments, the Evans blue derivative molecule is a pharmaceutically acceptable ester, amide, solvate or salt of the compound of Formula I. In other particular embodiments, the Evans blue derivative molecule is a salt of a pharmaceutically acceptable ester of the compound of Formula I. In other particular embodiments, the Evans blue derivative molecule is a salt of a pharmaceutically acceptable amide of the compound of Formula I. In other particular embodiments, the Evans blue derivative molecule is a solvate of a pharmaceutically acceptable ester the compound of Formula I. In other particular embodiments, the Evans blue derivative molecule is a solvate of a pharmaceutically acceptable amide the compound of Formula I. In other particular embodiments, the Evans blue derivative molecule is a solvate of a pharmaceutically acceptable salt of the compound of Formula I.

In a particular embodiment, L₁ in Formula I is —NH(CO)—, and L₃ is —NH(CO)CH₂—, that is, the compound of Formula I is a compound represented by the following Formula VII:

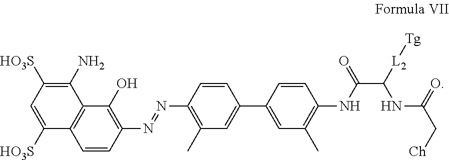

In a particular embodiment, the chelating group Ch in Formula I is selected from:

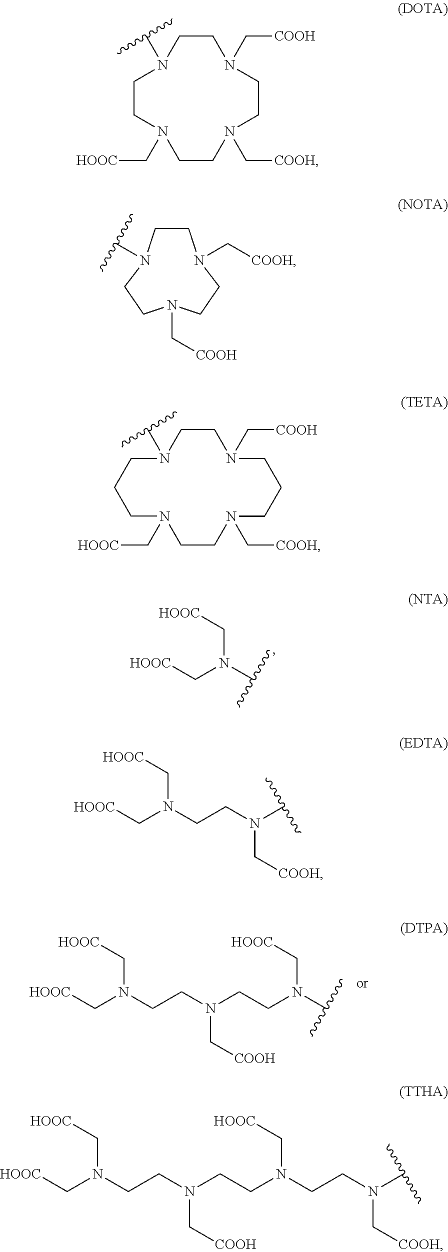

preferably the chelating group Ch in Formula I is

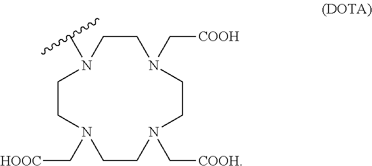 [SMILES: *N1CCN(CC(=O)O)CCN(CC(=O)O)CCN(CC(=O)O)CC1]

In a particular embodiment of the present application, the targeting group Tg in Formula I is a chemical group specifically targeting a certain biological target. In some embodiments, Tg is a chemical group capable of targeting a biological target selected from: somatostatin receptor (SSTR), prostate specific membrane antigen (PSMA), fibroblast activation protein (FAP), folate receptor (FR), epidermal growth factor receptor or integrin. In some particular embodiments, the targeting group Tg is selected from:

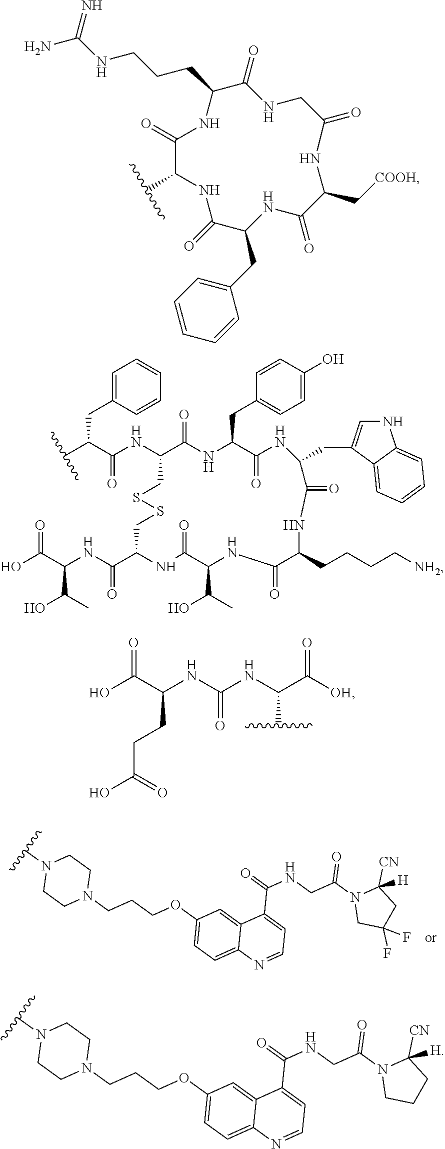

In a particular embodiment of the present application, the compound of Formula I is EB-PSMA, with the structural formula represented by the following Formula II:

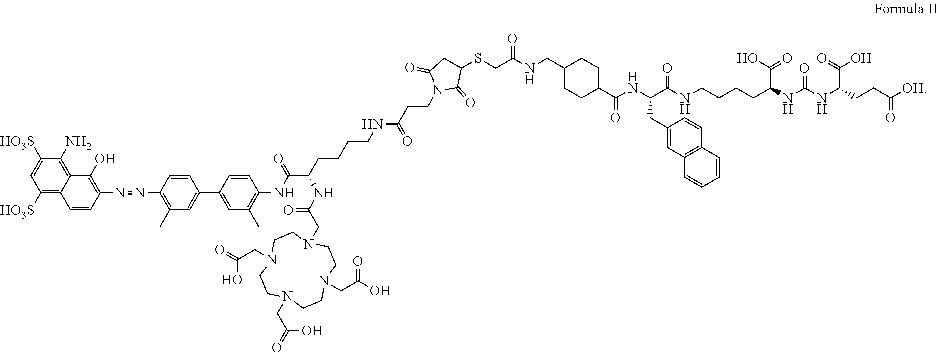 [SMILES: Cc1cc(-c2ccc(NC(=O)[C@H](CCCCNC(=O)CCN3C(=O)CC(SCC(=O)NCC4CCC(C(=O)N[C@@H](Cc5ccc6ccccc6c5)C(=O)NCCCC[C@H](NC(=O)N[C@@H](CCC(=O)O)C(=O)O)C(=O)O)CC4)C3=O)NC(=O)CN3CCN(CC(=O)O)CCN(CC(=O)O)CCN(CC(=O)O)CC3)c(C)c2)ccc1N=Nc1ccc2c(S(=O)(=O)O)cc(S(=O)(=O)O)c(N)c2c1O]

In a particular embodiment of the present application, the compound of Formula I is DOTA-EB-TATE, with the structural formula represented by the following Formula III:

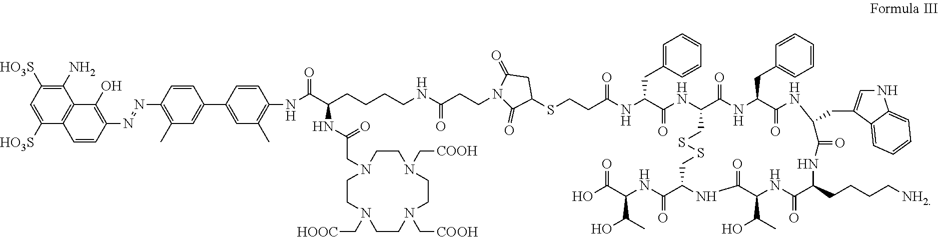 [SMILES: Cc1cc(-c2ccc(NC(=O)[C@@H](CCCCNC(=O)CCN3C(=O)CC(SCCC(=O)N[C@H](Cc4ccccc4)C(=O)N[C@H]4CSSC[C@@H](C(=O)N[C@H](C(=O)O)C(C)O)NC(=O)[C@H](C(C)O)NC(=O)[C@H](CCCCN)NC(=O)[C@@H](Cc5c[nH]c6ccccc56)NC(=O)[C@H](Cc5ccccc5)NC4=O)C3=O)NC(=O)CN3CCN(CC(=O)O)CCN(CC(=O)O)CCN(CC(=O)O)CC3)c(C)c2)ccc1/N=N/c1ccc2c(S(=O)(=O)O)cc(S(=O)(=O)O)c(N)c2c1O]

In a particular embodiment of the present application, the compound of Formula I is EB-FAPI, with the structural formula represented by the following Formula IV:

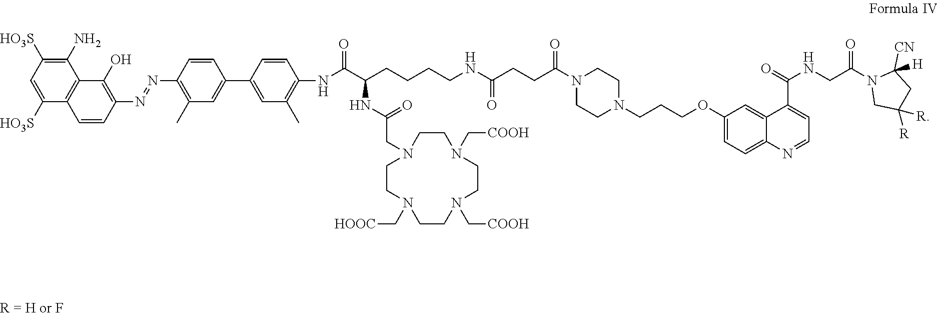

In a particular embodiment of the present application the compound of Formula I is NMEB-RGD, with the structural formula represented by the following Formula V:

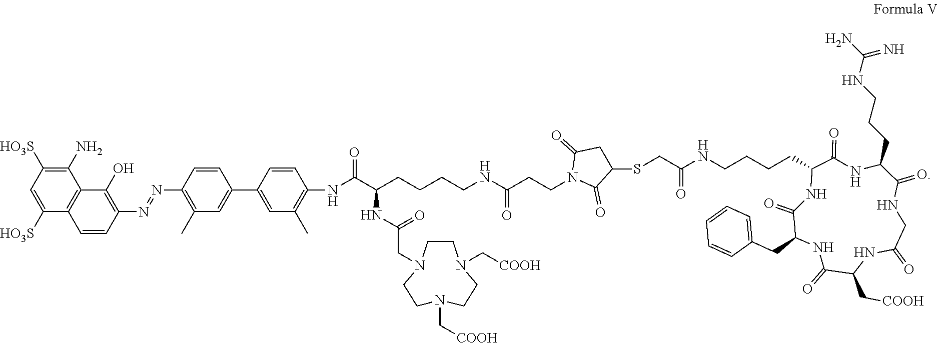 [SMILES: Cc1cc(-c2ccc(NC(=O)[C@@H](CCCCNC(=O)CCN3C(=O)CC(SCC(=O)NCCCC[C@H]4NC(=O)C[C@H](Cc5ccccc5)NC(=O)[C@H](CC(=O)O)NC(=O)CNC(=O)[C@H](CCCNC(=N)N)NC4=O)C3=O)NC(=O)CN3CCN(CC(=O)O)CCN(CC(=O)O)CC3)c(C)c2)ccc1/N=N/c1ccc2c(S(=O)(=O)O)cc(S(=O)(=O)O)c(N)c2c1O]

In a particular embodiment of the present application, the solution comprising radionuclides is taken out from the raw material bottle and then added to the reaction vessel, and after the solution comprising radionuclides is taken out, the raw material bottle is rinsed with a rinse liquid to extract the residual nuclide solution in the raw material bottle, and the rinsed solution is transferred into the reaction vessel to mix with the solution comprising radionuclides therein.

In a particular embodiment, the rinse solution is an aqueous solution, preferably selected from: a solution comprising a first stabilizer, a solution comprising a buffer salt, water, or sodium chloride injection.

In a preferred embodiment, the rinse solution is selected from: water for injection or sodium chloride injection.

In a preferred embodiment, the rinsing is repeated one or more times by using the rinse solution.

In a particular embodiment of the present application, the first stabilizer is one or more selected from the group consisting of gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin, preferably gentisic acid.

In a particular embodiment of the present application, a solution comprising a first stabilizer and a solution comprising a radionuclide are mixed in a reaction vessel, and after a given time, a solution comprising the Evans blue derivative molecule is added to the reaction vessel. The given time allows the first stabilizer to fully contact with the nuclide solution, so as to quench a large number of free radicals caused by radiolysis therein, thereby protecting the Evans blue derivative molecules subsequently added to the reaction system from being attacked by active free radicals, which is beneficial to improving the initial radiochemical purity of the final product.

In a preferred embodiment, the given time is 0.1-20 min, for example, it may be 0.1, 0.2, 0.3, 0.5, 0.8, 1, 1.5, 2, 2.5, 3, 3.5, 4, 4.5, 5, 5.5, 6, 7, 8, 9, 10, 11, 12, 13, 14, 15, 16, 17, 18, 19, or 20 min; more preferably 3-10 min, for example, it may be 3, 3.5, 4, 4.5, 5, 5.5, 6, 6.5, 7, 7.5, 8, 8.5, 9, 9.5, or 10 min.

In a particular embodiment, the solution comprising the Evans blue derivative molecules is added to the reaction phase solution to react with the radionuclide to obtain the radionuclide complex.

In a particular embodiment, the Evans blue derivative molecule (labeling precursor) solution is selected from compound solutions with concentrations of 0.05-10.0 mg/mL, and its preparation method is to dissolve the lyophilized powder of the labeling precursor in sterile water for injection or ethanol.

In a preferred embodiment, the radionuclide complex is ¹⁷⁷Lu-DOTA-EB-TATE.

In a particular embodiment, during the process of reacting the Evans blue derivative molecule with the radionuclide to obtain the radionuclide complex, the first stabilizer present is gentisic acid, and its concentration in the above reaction phase is 0.6-20.0 mg/mL, preferably 2-10 mg/mL, for example, 2, 2.5, 3, 3.5, 4, 4.5, 5, 5.5, 6, 6.5, 7, 7.5, 8, 8.5, 9, 9.5, or 10 mg/mL; most preferably 3.0-5.0 mg/mL, for example, 3.0, 3.2, 3.4, 3.5, 3.6, 3.8, 4.0, 4.2, 4.5, 4.8, or 5.0 mg/mL. When the concentration of gentisic acid in the reaction phase system exceeds a controlled range, the reaction rate will be greatly slowed down, which is not conducive to the entire synthesis process; and when the concentration of gentisic acid is lower than a controlled concentration, the radiation degradation impurities will be increased due to insufficient stabilizer concentration.

In a particular embodiment, in the reaction phase solution formed by reacting the Evans blue derivative molecule with the radionuclide, the molar ratio of the Evans blue derivative molecule to the radionuclide is 1.5-50, preferably 5-20, for example, 5, 8, 10, 12, 15, 18, or 20. The molar ratio refers to the ratio of the molar quantity of the Evans blue derivative molecule (labeling precursor) to the radionuclide in the reaction system. In the reaction phase solution, increase of the molar ratio is conducive to completely reaction of the radionuclide and improves the labeling rate, but the unlabeled labeling precursor will compete with the API in the body. However, a too low molar ratio will cause the API to lack a carrier and be easily lost by binding to other nonspecific targets in the body, thereby failing to achieve the desired therapeutic or diagnostic effect.

In the embodiment of the present application, the concentration of the reaction phase in the reaction phase solution may also be controlled. Theoretically, the higher the concentration of the reaction phase, the faster the labeling reaction rate, but at the same time the radiolysis effect caused by the radionuclide is also stronger. Therefore, the concentration of the reaction phase cannot be too high, however too low a concentration of the reaction phase will increase the reaction volume, thereby limiting the production of large quantities of nuclide complexes. For the preparation method according to the present application, in the reaction phase solution, the concentration of the Evans blue derivative molecule is 0.01-1.0 mg/mL, preferably 0.05-0.5 mg/mL, for example, it may be 0.05, 0.1, 0.15, 0.2, 0.25, 0.3, 0.35, 0.4, 0.45, or 0.5 mg/mL.

In an embodiment of the present application, in the step of performing a complex reaction of the Evans blue derivative molecule and the radionuclide, the reaction temperature and time period are controlled to achieve a reaction labeling rate of >90%, a chemical purity of >90%, and a radiochemical purity of >90%. In a particular embodiment, the reaction temperature is 50-100° C., preferably 60-80° C., for example, 60, 62, 65, 68, 70, 72, 75, 78, or 80° C.; and the reaction time period is 5-60 min, for example, 5, 10, 12, 15, 18, 20, 25, 30, 40, 50, or 60 min, preferably 10-30 min, most preferably 10-20 min, for example, 10, 11, 12, 13, 14, 15, 16, 17, 18, 19, or 20 min.

In a particular embodiment, after completion of reacting the Evans blue derivative molecule with the radionuclide for the above mentioned time period to form a complex, the second stabilizer is added. Particularly, the second stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin, preferably gentisic acid, ethanol or methionine.

In a particular embodiment, in the aqueous radiopharmaceutical solution, the concentration of the second stabilizer is 0-400 mg/mL, for example, 0, 0.5, 1, 2, 5, 10, 25, 50, 100, 150, 200, 250, 300, 350, or 400 mg/mL.

In an embodiment of the present application, the preparation method further comprises adding a buffer salt solution before reacting the Evans blue derivative molecule with the radionuclide. Preferably, the buffer salt solution is present in the solution comprising the first stabilizer.

In a particular embodiment, the buffer salt solution is selected from: acetate, citrate, phosphate or formate solution, preferably acetic acid-sodium acetate buffer salt solution.

The addition of buffer salt solution can adjust the pH value of the reaction system and control the pH value of the reaction phase system within a range of 3.5-6.0, for example, 3.5, 3.8, 4, 4.2, 4.4, 4.6, 4.8, 5, 5.5, or 6; the preferable pH value is 3.5-5. In a particular embodiment, the pH value of the final formulation solution is controlled to be 4-6, for example, 4, 4.2, 4.5, 4.8, 5, 5.2, 5.5, 5.8, or 6.

In a particular embodiment, the step of adding a solution comprising a second stabilizer into the reaction vessel after a given reaction time further comprises adding a cosolvent into the reaction vessel.

In a particular embodiment, the cosolvent is one or more selected from the group consisting of: polysorbate 20, polysorbate 40, polysorbate 60, polysorbate 80, poloxamer 188, polyoxyethylene castor oil, Span, ethanol, propylene glycol, glycerol, polyethylene glycol (average molecular weight of 200-8000), sorbitol, dimethyl sulfoxide, and sodium dodecyl sulfate; preferably polysorbate 80. In a particular embodiment, the cosolvent is added to have a concentration of 0.01-10 mg/mL in the pharmaceutical aqueous solution, preferably 0.05-1.0 mg/mL, for example, 0.05, 0.08, 0.1, 0.2, 0.3, 0.4, 0.5, 0.6, 0.7, 0.8, or 1.0 mg/mL.

In a particular embodiment, the step of adding a solution comprising a second stabilizer to the reaction vessel after a given reaction time further comprises adding a chelator for free nuclide to the reaction vessel, wherein the chelator may be selected from pentetic acid and a salt thereof, preferably pentetic acid. In a preferred embodiment, the chelator is added to have a concentration of 0.005-0.1 mg/mL in the pharmaceutical aqueous solution, for example, 0.005, 0.01, 0.02, 0.03, 0.04, 0.05, 0.06, 0.07, 0.08, 0.09, or 0.1 mg/mL.

In a particular embodiment, the preparation method according to the present application further comprises filtering the radiopharmaceutical aqueous solution for sterilization. In a particular embodiment, the radiopharmaceutical aqueous solution is filtered through a 0.22 μm filter membrane for sterilization.

In a particular embodiment, the preparation method according to the present application further comprises diluting the radioactive aqueous solution, preferably adding sodium chloride injection to dilute it for recovery.

In a preferred embodiment, the filtrating for sterilization and dilution are performed after adding the solution comprising the second stabilizer. The present application does not limit the order of the filtrating for sterilization and dilution steps, that is, filtrating for sterilization may be performed before the dilution, or dilution may be performed before filtrating through a filter membrane for sterilization, and then recovery may be performed.

In a particular embodiment, the present application provides a method for preparing a ¹⁷⁷Lu-DOTA-EB-TATE radiopharmaceutical aqueous solution in the following order:
a. transferring the nuclide solution comprising 500 mCi ¹⁷⁷Lu and hydrochloric acid from the raw material bottle to the reaction bottle;
b. adding 1 mL of a rinse solution comprising 2.0 M formic acid-sodium formate buffer and 50 mg/mL gentisic acid into the above raw material bottle to rinse the residual ¹⁷⁷Lu solution in the raw material bottle;
c. transferring the mixed solution in the rinsed raw material bottle into the reaction bottle;
d. adding 3 mL of water for injection into the above raw material bottle to rinse the raw material bottle;
e. transferring the mixed solution in the rinsed raw material bottle into the reaction bottle;
f. allowing the reaction bottle containing the above solution to stand at room temperature for 10 min;
g. adding 0.5 mL of DOTA-EB-TATE solution into the reaction bottle;
h. raising the temperature of the reaction bottle to 90° C. to react for 15 min;
i. cooling the reaction bottle after completion of the reaction, and adding 10 mL of a mixed solution comprising 0.5 mg/mL pentetic acid, 45 mg/mL gentisic acid and 2.0 mg/mL polysorbate 80 to the reaction bottle;
j. filtering the resulting solution through a 0.22 μm filter membrane for sterilization;
k. diluting the resulting solution with 35 mL of sodium chloride injection; and
l. recovering the resulting product.

### EXAMPLES

The experimental methods used in the following Examples are all conventional methods unless otherwise specified.

The precursor EB-PSMA used in the following Examples was synthesized according to the literature method (Bioconjugate Chem. 2018, 29, 3213-3221).

The precursor DOTA-EB-TATE used in the following Examples was synthesized according to the literature method (Theranostics. 2018; 8: 735-745).

The precursor EB-FAPI used in the following Examples was synthesized according to the literature method (Theranostics. 2022; 12(1): 422-433).

The gentisic acid used in the following Examples was purchased from Chengdu Phytopurify Technology Development Co., Ltd., and the pentetic acid was purchased from Jiangxi Alpha Hi-Tech Pharmaceutical Co., Ltd.

Other materials and reagents are commercially available, unless otherwise specified.

### Example 1: Selection of a Stabilizer in a Pharmaceutical Aqueous Solution

### Prescription (1): Preparation of [¹⁷⁷Lu]Lu-DOTA-EB-TATE Pharmaceutical Aqueous Solution

Preparation of reaction phase solution: 10 mCi of carrier-free lutetium chloride [¹⁷⁷Lu] solution (about 10 μL), 20 μL of formic acid-sodium formate buffer salt solution (comprising 50 mg/mL gentisic acid), and 60 μL of water for injection were added into a reaction vessel to mix well, then allowing the mixed solution stand at room temperature for 3 min. After that, 10 μL of DOTA-EB-TATE precursor solution was added to the reaction vessel to mix well to obtain a mixed solution as the reaction phase solution.

Heating reaction and cooling: the above reaction phase solution was placed in a heater preheated to 90° C. for reaction for 15 min. After the reaction was completed, the reaction vessel was taken out to cool for 15 min.

Adding auxiliary solution and dilution: after the reaction phase solution is cooled to room temperature, 200 μL of auxiliary solution comprising 45 mg/mL gentisic acid, 0.5 mg/mL pentetic acid and 2.0 mg/mL polysorbate 80 was added to the reaction vessel. Finally, sodium chloride injection was added to the reaction vessel to dilute the total volume to 1.0 mL to obtain the final formulation solution.

The final formulation solution comprises 10 mg/mL gentisic acid, 0.1 mg/mL pentetic acid, and 0.4 mg/mL polysorbate 80, wherein the activity concentration of the API molecule [¹⁷⁷Lu]Lu-DOTA-EB-TATE at the calibration time is 10 mCi/mL, and the calibration time refers to the time when production ends (To). The formulation solution was stored in a stabilization box with a storage temperature of 32° C. and a storage humidity of 60% RH.

The radiochemical purity of the formulation solution was 100% as determined by Radio-HPLC at time T₀, and was 100% as determined by ITLC at time T₀.

At T₀+48 h, 150 μL of the formulation solution was taken out from the stabilization box for stability testing. The radiochemical purity of the formulation solution was 94% as determined by Radio-HPLC, and was 100% as determined by ITLC.

At T₀+72 h, 150 μL of the formulation solution was taken out from the stabilization box for stability testing. The radiochemical purity of the preparation solution was 92% as determined by Radio-HPLC, and was 100% as determined by ITLC.

### Prescription (2): Preparation of [¹⁷⁷Lu]Lu-DOTA-EB-TATE Pharmaceutical Aqueous Solution

Preparation of reaction phase solution: 10 mCi of carrier-free lutetium chloride [¹⁷⁷Lu] solution (about 10 L), 20 μL of formic acid-sodium formate buffer salt solution (comprising 50 mg/mL gentisic acid), and 160 μL of water for injection were added into a reaction vessel to mix well, then allowing the mixed solution stand at room temperature for 3 min. After that, 10 μL of DOTA-EB-TATE precursor solution was added to the reaction vessel to mix well to obtain a mixed solution as the reaction phase solution.

Heating reaction and cooling: the above reaction phase solution was placed in a heater preheated to 65° C. for reaction for 40 min. After the reaction was completed, the reaction vessel was taken out to cool for 15 min.

Adding auxiliary solution and dilution: after the reaction phase solution is cooled to room temperature, 100 μL of auxiliary solution comprising 0.3 mg/mL pentetic acid and 1.0 mg/mL polysorbate 80 was added to the reaction vessel. Then 50 mg of anhydrous ethanol was added, and finally sodium chloride injection was added to the reaction vessel to dilute the total volume to 1.0 mL to obtain the final formulation solution.

The final formulation solution comprises 1 mg/mL gentisic acid, 50 mg/mL ethanol, 0.03 mg/mL pentetic acid, and 0.1 mg/mL polysorbate 80, wherein the activity concentration of the API molecule [¹⁷⁷Lu]Lu-DOTA-EB-TATE at the calibration time is 10 mCi/mL, and the calibration time refers to the time when production ends (To). The formulation solution was stored in a stabilization box with a storage temperature of 32° C. and a storage humidity of 60% RH.

The radiochemical purity of the formulation solution was 100% as determined by Radio-HPLC at time T₀, and was 100% as determined by ITLC at time T₀.

At T₀+48 h, 150 μL of the formulation solution was taken out from the stabilization box for stability testing. The radiochemical purity of the formulation solution was 93% as determined by Radio-HPLC, and was 100% as determined by ITLC.

At T₀+72 h, 150 μL of the formulation solution was taken out from the stabilization box for stability testing. The radiochemical purity of the preparation solution was 92% as determined by Radio-HPLC, and was 99% as determined by ITLC.

### Prescription (3): Preparation of [¹⁷⁷Lu]Lu-EB-FAPI Pharmaceutical Aqueous Solution

Preparation of reaction phase solution: 20 mCi of carrier-free lutetium chloride [¹⁷⁷Lu] solution (about 20 L), 20 μL of ammonium acetate buffer salt solution (comprising 50 mg/mL gentisic acid), and 40 μL of water for injection were added into a reaction vessel to mix well, then allowing the mixed solution stand at room temperature for 10 min. After that, 20 μL of EB-FAPI precursor solution was added to the reaction vessel to mix well to obtain a mixed solution as the reaction phase solution, wherein EB-FAPI precursor is a compound of Formula IV, and R═H.

Heating reaction and cooling: the above reaction phase solution was placed in a heater preheated to 95° C. for reaction for 30 min. After the reaction was completed, the reaction vessel was taken out to cool for 15 min.

Adding auxiliary solution and dilution: after the reaction phase solution is cooled to room temperature, 500 μL of auxiliary solution comprising 20 mg/mL methionine, 4.0 mg/mL gentisic acid, 0.2 mg/mL pentetic acid and 0.8 mg/mL polysorbate 80 was added to the reaction vessel.

Finally, sodium chloride injection was added to the reaction vessel to dilute the total volume to 1.0 mL to obtain the final formulation solution.

The final formulation solution comprises 3.0 mg/mL gentisic acid, 10 mg/mL methionine, 0.1 mg/mL pentetic acid, and 0.4 mg/mL polysorbate 80, wherein the activity concentration of the API molecule [¹⁷⁷Lu]Lu-EB-FAPI at the calibration time is 20 mCi/mL, and the calibration time refers to the time when production ends (To). The formulation solution was stored in a stabilization box with a storage temperature of 32° C. and a storage humidity of 60% RH.

The radiochemical purity of the formulation solution was 98% as determined by Radio-HPLC at time T₀, and was 100% as determined by ITLC at time T₀.

At T₀+48 h, 150 μL of the formulation solution was taken out from the stabilization box for stability testing. The radiochemical purity of the formulation solution was 96% as determined by Radio-HPLC, and was 100% as determined by ITLC.

At T₀+72 h, 150 μL of the formulation solution was taken out from the stabilization box for stability testing. The radiochemical purity of the preparation solution was 93% as determined by Radio-HPLC, and was 100% as determined by ITLC.

Prescription (4): Preparation of [¹⁷⁷Lu]Lu-EB-PSMA pharmaceutical aqueous solution Preparation of reaction phase solution: 10 mCi of carrier-free lutetium chloride [¹⁷⁷Lu] solution (about 10 L), 20 μL of formic acid-sodium formate buffer salt solution (comprising 50 mg/mL gentisic acid), and 60 μL of water for injection were added into a reaction vessel to mix well, then allowing the mixed solution stand at room temperature for 3 min. After that, 10 μL of EB-PSMA precursor solution was added to the reaction vessel to mix well to obtain a mixed solution as the reaction phase solution.

Heating reaction and cooling: the above reaction phase solution was placed in a heater preheated to 80° C. for reaction for 15 min. After the reaction was completed, the reaction vessel was taken out to cool for 15 min.

Adding auxiliary solution and dilution: after the reaction phase solution is cooled to room temperature, 100 μL of auxiliary solution comprising 30 mg/mL ascorbic acid, 1.0 mg/mL pentetic acid and 4.0 mg/mL polysorbate 80 was added to the reaction vessel. Finally, sodium chloride injection was added to the reaction vessel to dilute the total volume to 1.0 mL to obtain the final formulation solution.

The final formulation solution comprises 1.0 mg/mL gentisic acid, 3.0 mg/mL ascorbic acid, 0.1 mg/mL pentetic acid and 0.4 mg/mL polysorbate 80, wherein the activity concentration of the API molecule [¹⁷⁷Lu]Lu-EB-PSMA at the calibration time is 10 mCi/mL, and the calibration time refers to the time when production ends (T₀). The formulation solution was stored in a stabilization box with a storage temperature of 32° C. and a storage humidity of 60% RH.

The radiochemical purity of the formulation solution was 93% as determined by Radio-HPLC at time T₀.

At T₀+48 h, 150 μL of the formulation solution was taken out from the stabilization box, and the radiochemical purity of the formulation solution was 81% as determined by Radio-HPLC.

At T₀+72 h, 150 μL of the formulation solution was taken out from the stabilization box, and the radiochemical purity of the preparation solution was 66% as determined by Radio-HPLC.

Analysis of experimental results: the pharmaceutical aqueous solutions prepared by respectively using gentisic acid, gentisic acid and ethanol, and gentisic acid and methionine as stabilizer in the above prescriptions (1) to (3) can obtain more than 90% radiochemical purity of API in 48 h and 72 h, which is significantly better than the selection of ascorbic acid as a stabilizer in prescription (4). This indicates that the pharmaceutical aqueous solutions provided by the present application can maintain better stability.

### Example 2: Control of Reaction Temperature and Time

Preparation of reaction phase solution: same as prescription (3).

Heating reaction and cooling: the above reaction phase solution was placed at room temperature or in heaters preheated to different temperatures to react for 120 min.

The temperatures investigated included room temperature, 50° C., 60° C., 70° C., 80° C., 90° C., 95° C., and 100° C.

Labeling rate detection: at different time points during the reaction, 5 μCi of the reaction phase solution was taken out for ITLC detection. Before and after sampling, the reaction phase solution was placed in a heater for reaction, that is, the sampling process did not affect the continuous progress of the reaction. The labeling rate (ITLC) was calculated=radioactivity by chelating to the precursor molecule÷total radioactivity. The time periods investigated included 1, 5, 10, 30, 45, 60, 90, and 120 min.

For labeling rate, reaction temperature and reaction time are two complementary process conditions. “Reacting at a lower temperature for a longer time” or “Reacting at a higher temperature for a shorter time” can both make the reaction labeling rate (ITLC) >99%; at this time, we believe that the labeling reaction has been completed. However, considering that high temperature will promote the formation of chemical impurities and radiochemical impurities, and a reaction time of more than 60 min is not conducive to the control of the process, thus the reaction temperature is controlled at 50-100° C., and the reaction time is controlled at 5-60 min. Preferably, the reaction temperature is controlled at 60-80° C., and the reaction time is controlled at 10-30 min.

### Example 3: Control of the Feeding Ratio

Preparation of reaction phase solution: 10 mCi of carrier-free lutetium chloride [¹⁷⁷Lu] solution (about 10 L) and 20 μL of formic acid-sodium formate buffer solution (comprising 50 mg/mL gentisic acid) were added into a reaction vessel, allowing the mixed solution to stand at room temperature for 3 min; then continuing to add different volumes of injection water and DOTA-EB-TATE precursor solution into the reaction vessel, so that the molar ratio of DOTA-EB-TATE to lutetium chloride [¹⁷⁷Lu] in the reaction phase is 1, 1.5, 2, 3, 5, 10, 15, 30 and 50 respectively. Finally, the total volume of the solution is 0.1 mL, and the solution is mixed evenly as the reaction phase solution.

Heating reaction and cooling: same as prescription (1).

Adding auxiliary solution and dilution: same as prescription (1).

Labeling rate detection: 5 μCi of the reaction phase solution was taken for ITLC detection, and the labeling rate (ITLC) was calculated=radioactivity by chelating to the precursor molecule÷total radioactivity.

When the feeding molar ratio of the precursor molecule to the nuclide is 1.5-50, the labeling rate is ≥95%; when the feeding molar ratio of the precursor molecule to the nuclide is 3-50, the labeling rate is ≥99%, and at this time, we believe that the labeling reaction has been completed.

### Example 4: Control of Quenching Time

Preparation of reaction phase solution A: 10 mCi of carrier-free lutetium chloride [¹⁷⁷Lu] solution (about 10 L), 10 μL of EB-FAPI precursor solution, 60 μL of water for injection, and 20 μL of acetic acid-sodium acetate buffer solution (comprising 50 mg/mL gentisic acid) were added in a reaction vessel in sequence to mix well. The mixed solution is reaction phase solution A, and the precursor EB-FAPI is a compound of Formula IV, wherein R═H.

Preparation of reaction phase solution B: 10 mCi of carrier-free lutetium chloride [¹⁷⁷Lu] solution, 20 μL of acetic acid-sodium acetate buffer solution (comprising 50 mg/mL gentisic acid), and 60 μL of water for injection were added into a reaction vessel to mix well, then allowing the mixed solution to stand at room temperature for 3 min (i.e., quenching time). Then, 10 μL of EB-FAPI precursor solution was added to the reaction vessel to mix well. The mixed solution is the reaction phase solution B, and the precursor EB-FAPI is a compound of Formula IV, wherein R═H.

Preparation of reaction phase solution C: except for the quenching time of 15 min, the rest is the same as reaction phase solution B.

Heating reaction and cooling: same as prescription (1).

Adding auxiliary solution and dilution: same as prescription (1).

Radiochemical purity detection: after completion of adding the auxiliary solution for dilution, 150 μL of the reaction phase solution was immediately taken for HPLC assay, and the radiochemical purity (HPLC) was calculated=peak area of the labeled compound÷total peak area.

The radiochemical purity of the reaction phase solutions A, B and C are 93%, 99% and 99% respectively. The experimental results show that before adding the precursor solution, the mixed solution comprising the radionuclide solution, the buffer salt and the first stabilizer is allowed to stand for a short period of time (quenching time) followed by adding the precursor molecule to react, such a feeding sequence can significantly improve the initial radiochemical purity of the API. Because after a period of quenching time, the first stabilizer is fully in contact with the nuclide solution, so that a large number of free radicals generated in the solution due to high radioactivity are quenched by the first stabilizer, thereby reducing the damage of free radicals to the labeled precursor molecules when the precursor molecules are added subsequently. The quenching time depends on the type and initial activity of the nuclide. Generally speaking, the quenching time is controlled in a range of 0.1-20 min, preferably 3-10 min.

### Example 5: Labeling of [⁶⁸Ga]Ga-EB-FAPI

Preparation of reaction phase solution: a commercially available gallium-germanium generator was rinsed with 0.1M hydrochloric acid to obtain ⁶⁸Ga hydrochloric acid solution, 5 mCi of ⁶⁸Ga hydrochloric acid solution was taken to add in a reaction vessel, adding 20 μL of sodium acetate solution (comprising 50 mg/mL gentisic acid) and 150 μL water for injection to mix well, allowing the mixed solution to stand at room temperature for 6 min, then adding 5 μL of EB-FAPI precursor solution to the reaction vessel, and adding water for injection to make a total volume of the solution 0.3 mL and mix well. The mixed solution is the reaction phase solution, and the precursor EB-FAPI is a compound of Formula IV, wherein R═H.

Heating reaction and cooling: the above reaction phase solution was placed in a heater preheated to 95° C. for reaction for 30 min. After completion of the reaction, the reaction vessel was taken out to cool for 5 min.

Purification: the reaction phase solution was purified by using a C18 column, then eluting the labeled complex into the product bottle with 0.4 mL of anhydrous ethanol.

Adding auxiliary solution and dilution: 200 μL of auxiliary solution comprising 45 mg/mL gentisic acid, 0.5 mg/mL pentetic acid and 2.0 mg/mL polysorbate 80 was added to the product bottle, finally adding sodium chloride injection to the product bottle to dilute a total volume to 1.0 mL to obtain the final formulation solution.

The final formulation solution comprises 9 mg/mL gentisic acid, 40% ethanol by volume (i.e., 315.6 mg/mL), 0.1 mg/mL pentetic acid and 0.4 mg/mL polysorbate 80, wherein the activity concentration of the API molecule [¹⁷⁷Lu]Lu-EB-FAPI at the calibration time is 5 mCi/mL, and the calibration time refers to the time when production ends (To). The formulation solution was stored in a stabilization box; the storage temperature was set at 25° C., and the storage humidity was set at 60% RH.

The radiochemical purity of the formulation solution was 99% as determined by Radio-HPLC at time T₀, and was 100% as determined by ITLC at time T₀.

At T₀+5 h, 150 μL of the formulation solution was taken out from the stabilization box. The radiochemical purity of the formulation solution was 93% as determined by Radio-HPLC, and was 100% as determined by ITLC.

Although the present application has been disclosed above by examples, it is not intended to limit the present application. Any skilled person with ordinary knowledge in this technical field may make some changes and modifications without departing from the spirit and scope of the present application. Therefore, the protection scope of the present application shall be determined by the appended claims.

1-63. (canceled)

64. A radiopharmaceutical aqueous solution, comprising: a complex formed by a compound represented by Formula I or a pharmaceutically acceptable ester, amide, solvate or salt thereof, or a compound represented by Formula I or a salt of a pharmaceutically acceptable ester thereof, or a compound represented by Formula I or a salt of a pharmaceutically acceptable amide thereof, or a compound represented by Formula I or a solvate of a pharmaceutically acceptable ester thereof, or a compound represented by Formula I or a solvate of a pharmaceutically acceptable amide thereof, or a compound represented by Formula I or a solvate of a pharmaceutically acceptable salt thereof, and a radioactive metal nuclide, as well as a stabilizer which is present at a total concentration of 0.5-400 mg/mL, 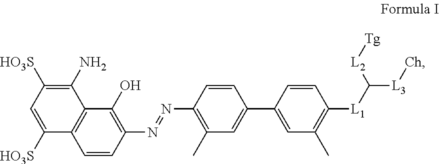
wherein
L₁ is —(CH₂)ₘ—, wherein m is an integer from 0 to 12, wherein each CH₂ may be individually replaced with —O—, —NH(CO)— or —(CO)NH—, providing no two adjacent CH₂ groups are replaced;
L₂ is a C₁-C₆₀ linking group, optionally including —O—, —S—, —S(O)—, —S(O)₂—, —N(R)—, —C(═O)—, —C(═O)O—, —OC(═O)—, —N(R)C(═O)—, —C(═O)N(R)—, —OC(═O)O—, —N(R)C(═O)O—, —OC(═O)N(R)—, 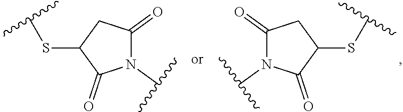
wherein each R is H or C₁-C₆ alkyl;
L₃ is —(CH₂)ₙ—, wherein n is an integer from 0 to 12, wherein each CH₂ may be individually replaced with —O—, —NH(CO)— or —(CO)NH—, providing no two adjacent CH₂ groups are replaced;
Ch is a chelating group;
Tg is a targeting group;
preferably, L₁ is —NH(CO)—, and L₃ is —NH(CO)CH₂—;
preferably, Ch is selected from: 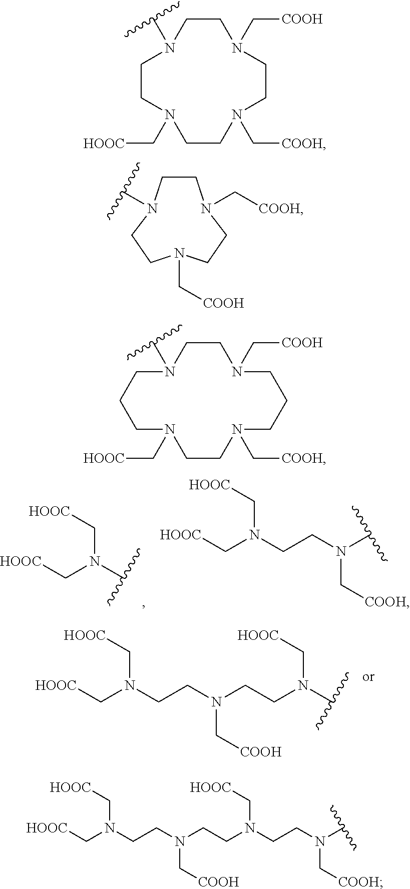
preferably, Tg is selected from a chemical group capable of targeting somatostatin receptor (SSTR), prostate-specific membrane antigen (PSMA), fibroblast activation protein (FAP), folate receptor (FR), epidermal growth factor receptor or integrin, further preferably,
Tg is selected from: 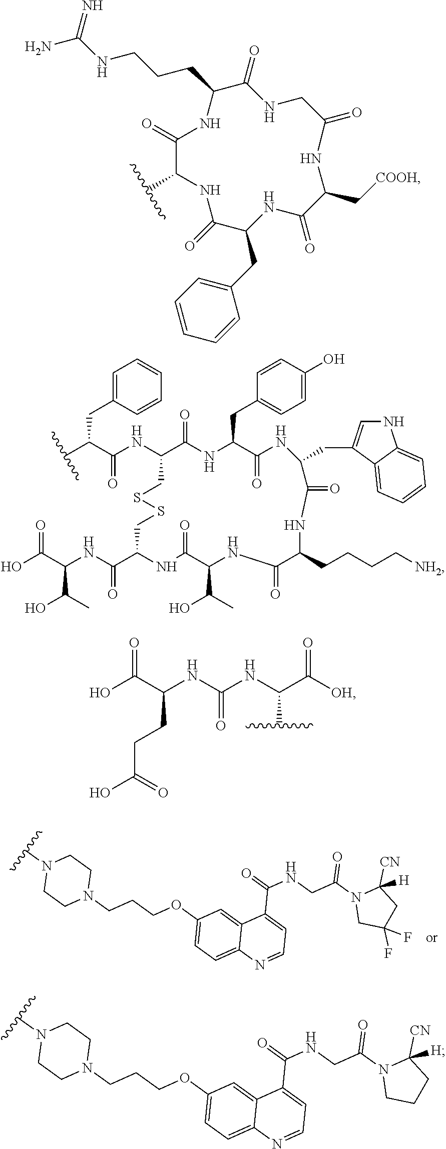
preferably, the radioactive metal nuclide is selected from: ¹⁷⁷Lu, ^{99m}Tc, ⁶⁸Ga, ⁶⁴Cu, ⁶⁷Cu, ¹¹¹In, ⁸⁶Y, ⁹⁰Y, ⁸⁹Zr, ¹⁸⁶Re, ¹⁸⁸Re, ¹⁵³Sm, ⁸²Rb, ¹⁶⁶Ho, ²²⁵Ac, ²¹²Pb, ²¹³Bi, ²¹²Bi or ²²⁷Th.

65. The pharmaceutical aqueous solution according to claim 64, wherein Formula I is Formula II, Formula III, Formula IV or Formula V,

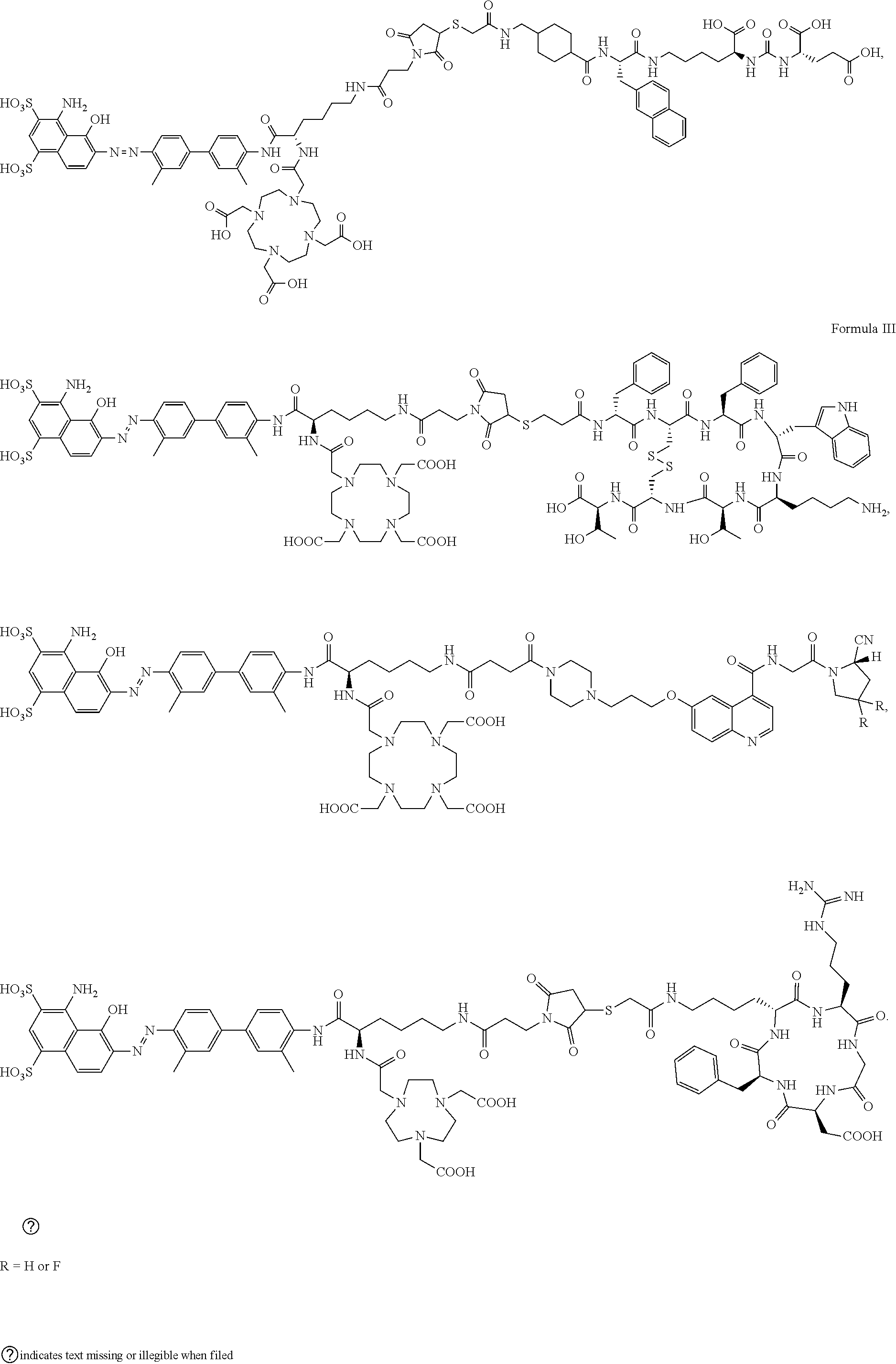

66. The pharmaceutical aqueous solution according to claim 64, wherein the stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin.

67. The pharmaceutical aqueous solution according to claim 64, wherein the stabilizer is respectively added during the reaction of forming the complex and after completion of the reaction,
preferably, the stabilizer added during the reaction of forming the complex is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin;
the stabilizer added after completion of the reaction of forming the complex is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin.

68. The pharmaceutical aqueous solution according to claim 67, wherein the stabilizer added during and after the reaction of forming the complex is gentisic acid, and the gentisic acid is present in the pharmaceutical aqueous solution at a total concentration of 0.1-10 mg/mL, preferably, the gentisic acid is present in the pharmaceutical aqueous solution at a total concentration of 0.5-5 mg/mL; or,
the stabilizer added during the reaction of forming the complex is gentisic acid, and the stabilizer added after completion of the reaction of forming the complex is ethanol; gentisic acid is present at a concentration of 0.1-10 mg/mL and ethanol is present at a concentration of 0-400 mg/mL in the pharmaceutical aqueous solution, preferably, the gentisic acid is present in the pharmaceutical aqueous solution at a concentration of 0.5-5 mg/mL; or,
the stabilizer added during the reaction of forming the complex is gentisic acid, and the stabilizer added after completion of the reaction of forming the complex is methionine; gentisic acid is present at a concentration of 0.1-10 mg/mL and methionine is present at a concentration of 0-50 mg/mL in the pharmaceutical aqueous solution, preferably, the gentisic acid is present in the pharmaceutical aqueous solution at a concentration of 0.5-5 mg/mL.

69. The pharmaceutical aqueous solution according to claim 64, further comprising a buffer solution, wherein the buffer solution may be selected from: acetate, citrate, phosphate or formate solution; and the buffer salt of the buffer solution is present in the pharmaceutical aqueous solution at a concentration of 0.005-0.5M.

70. The pharmaceutical aqueous solution according to claim 64, further comprising a cosolvent, wherein the cosolvent is one or more selected from the group consisting of: polysorbate 20, polysorbate 40, polysorbate 60, polysorbate 80, poloxamer 188, polyoxyethylene castor oil, Span, ethanol, propylene glycol, glycerol, polyethylene glycol (average molecular weight of 200-8000), sorbitol, dimethyl sulfoxide, and sodium dodecyl sulfate, preferably, the cosolvent is polysorbate 80.

71. The pharmaceutical aqueous solution according to claim 64, further comprising a chelator for free nuclide, wherein the chelator for free nuclide may be selected from pentetic acid and a salt thereof; and the chelator for free nuclide is present in the pharmaceutical aqueous solution at a concentration of 0.005-0.1 mg/mL.

72. A method for preparing a radiopharmaceutical aqueous solution, wherein the radiopharmaceutical aqueous solution comprises a complex formed by a radionuclide and an Evans blue derivative molecule, wherein the method comprises the following steps:
mixing a solution comprising a first stabilizer with a solution comprising a radionuclide in a reaction vessel;
after a given time, adding a solution comprising the Evans blue derivative molecule to the reaction vessel, wherein the given time is 0.1-20 min;
reacting the Evans blue derivative molecule with the radionuclide to obtain the radionuclide complex;
after a given reaction time, adding a solution comprising a second stabilizer to the reaction vessel;
recovering the obtained radiopharmaceutical aqueous solution;
wherein the Evans blue derivative molecule is a compound represented by Formula I or a pharmaceutically acceptable ester, amide, solvate or salt thereof, or a compound represented by Formula I or a salt of a pharmaceutically acceptable ester thereof, or a compound represented by Formula I or a salt of a pharmaceutically acceptable amide thereof, or a compound represented by Formula I or a solvate of a pharmaceutically acceptable ester thereof, or a compound represented by Formula I or a solvate of a pharmaceutically acceptable amide thereof, or a solvate of a compound represented by Formula I or a pharmaceutically acceptable salt thereof, 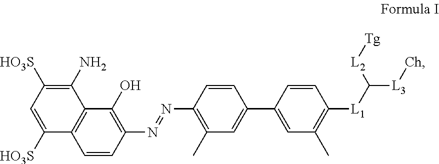
wherein
L₁ is —(CH₂)ₘ—, wherein m is an integer from 0 to 12, wherein each CH₂ may be individually replaced with —O—, —NH(CO)— or —(CO)NH—, providing no two adjacent CH₂ groups are replaced;
L₂ is a C₁-C₆₀ linking group, optionally including —O—, —S—, —S(O)—, —S(O)₂—, —N(R)—, —C(═O)—, —C(═O)O—, —OC(═O)—, —N(R)C(═O)—, —C(═O)N(R)—, —OC(═O)O—, —N(R)C(═O)O—, —OC(═O)N(R)—, 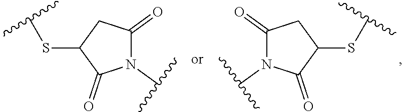
wherein each R is H or C₁-C₆ alkyl;
L₃ is —(CH₂)ₙ—, wherein n is an integer from 0 to 12, wherein each CH₂ may be individually replaced with —O—, —NH(CO)— or —(CO)NH—, providing no two adjacent CH₂ groups are replaced;
Ch is a chelating group;
Tg is a targeting group;
preferably, Ch in Formula I is selected from: 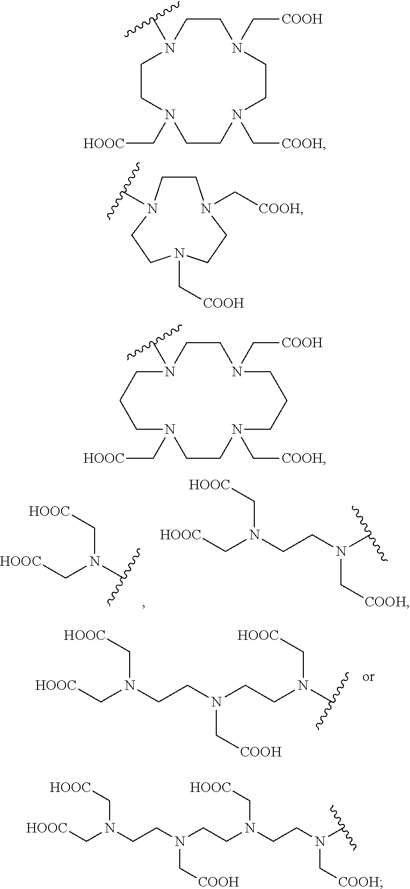
preferably, Tg in Formula I is selected from a chemical group capable of targeting somatostatin receptor (SSTR), prostate-specific membrane antigen (PSMA), fibroblast activation protein (FAP), folate receptor (FR), epidermal growth factor receptor or integrin;
preferably, the radionuclide is selected from: ¹⁷⁷Lu, ^{99m}Tc, ⁶⁸Ga, ⁶⁴Cu, ⁶⁷Cu, ¹¹¹In, ⁸⁶Y ⁹⁰Y, ⁸⁹Zr, ¹⁸⁶Re, ¹⁸⁸Re, ¹⁵³Sm, ⁸²Rb, ¹⁶⁶Ho, ²²⁵Ac, ²¹²Pb, ²¹³Bi ²¹²Bi or ²²⁷Th.

73. The method according to claim 72, wherein the Evans blue derivative molecule is selected from the compounds represented by Formula II, Formula III, Formula IV or Formula V,

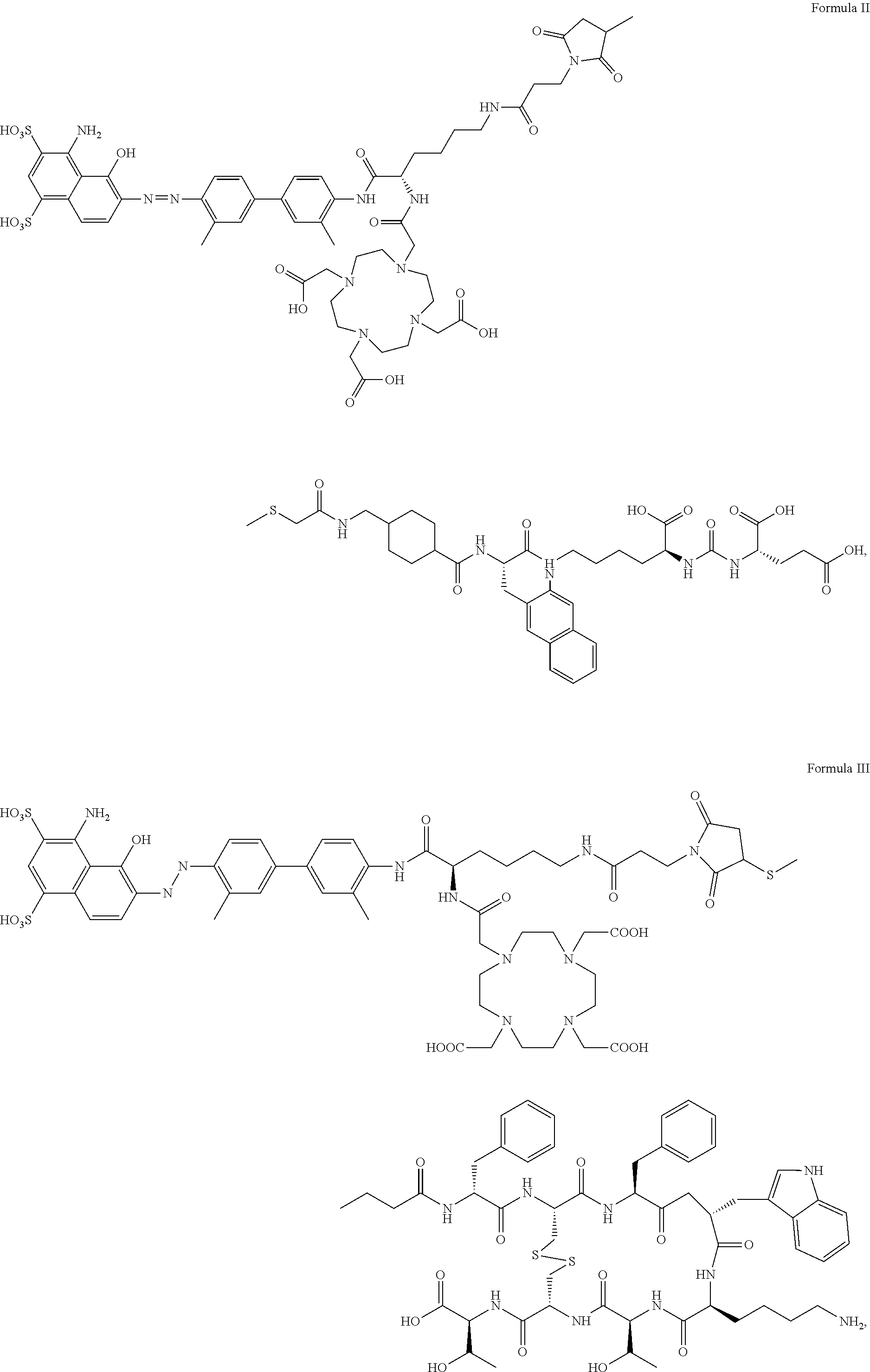

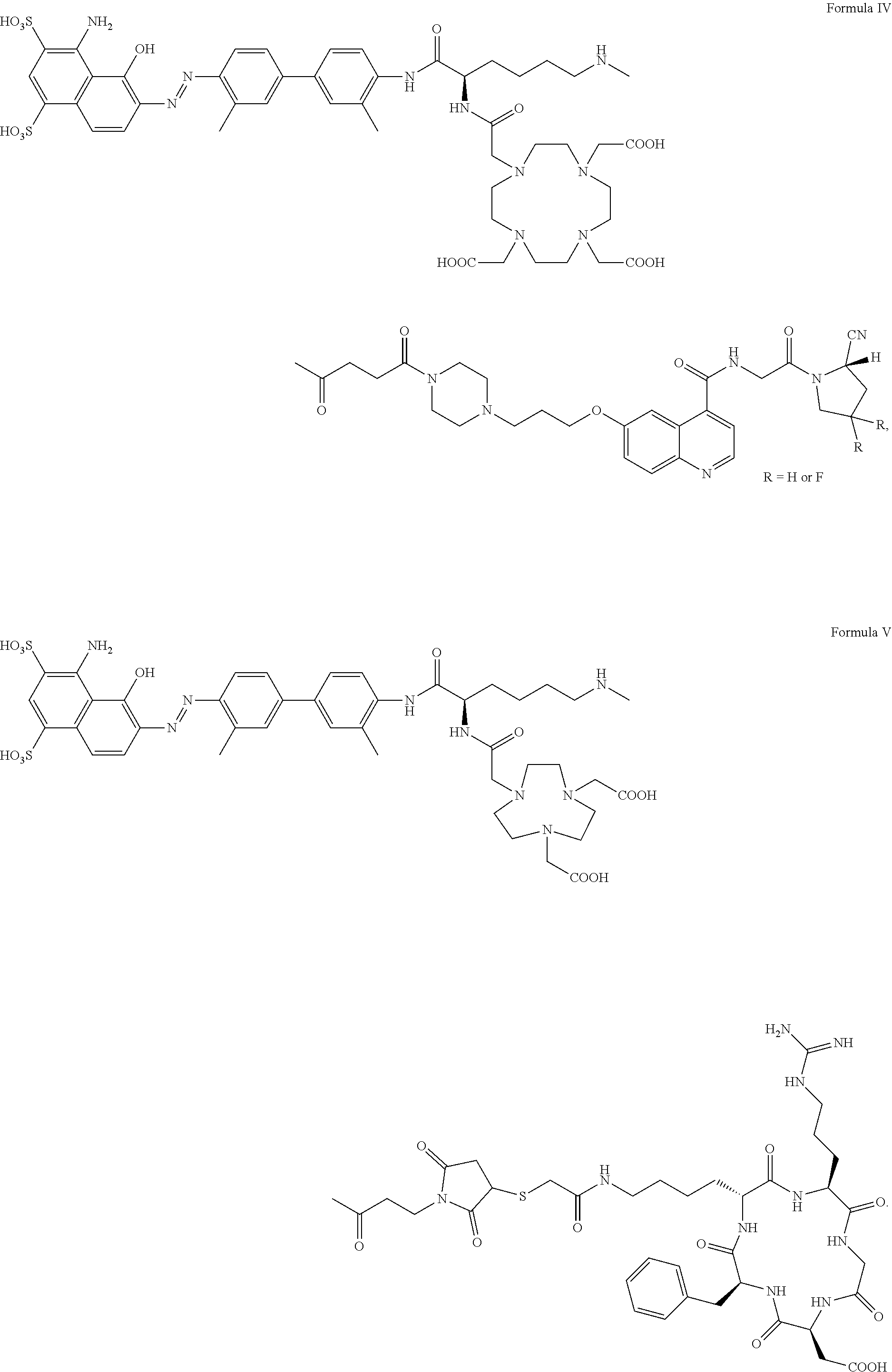

74. The method according to claim 72, wherein in the step of adding the solution comprising the Evans blue derivative molecule to the reaction vessel after a given time, the given time is 3-10 min.

75. The method according to claim 72, wherein the solution comprising the radionuclide is taken out from the raw material bottle and then added to the reaction vessel, and the method further comprises:
rinsing the raw material bottle with a rinse solution, and the rinsed solution is transferred into the reaction vessel to be mixed with the solution comprising the radionuclide;
preferably, the rinse solution is an aqueous solution;
preferably, the rinse solution is selected from: a solution comprising a first stabilizer, a solution comprising a buffered salt, water or sodium chloride injection;
further preferably, rinsing with the rinse solution is repeated one or more times.

76. The method according to claim 72, wherein in the step of reacting the Evans blue derivative molecule with the radionuclide, the molar ratio of the Evans blue derivative molecule to the radionuclide is 1.5-50, preferably, the molar ratio of the Evans blue derivative molecule to the radionuclide is 5-20.

77. The method according to claim 72, wherein in the step of reacting the Evans blue derivative molecule with the radionuclide, the reaction temperature is 50-100° C., preferably 60-80° C., and the reaction time is 5-60 min, preferably 10-30 min.

78. The method according to claim 72, wherein the first stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin; the second stabilizer is one or more selected from the group consisting of: gentisic acid and a salt thereof, ascorbic acid and a salt thereof, histidine, cysteine and a salt thereof, methionine, selenomethionine, thiosulfate, maltose, inositol, benzyl alcohol, trehalose, povidone, nicotinamide, ethanol, curcumin, and melatonin;
preferably, in the step of reacting the Evans blue derivative molecule with the radionuclide, the concentration of the first stabilizer in the reaction phase solution is 0.6-20.0 mg/mL; in the radiopharmaceutical aqueous solution, the concentration of the second stabilizer is 0-400 mg/mL.

79. The method according to claim 72, wherein a buffer salt solution is added before reacting the Evans blue derivative molecule with the radionuclide, preferably, the buffer salt solution is present in the solution comprising the first stabilizer, further preferably, the buffer salt solution is selected from: acetate, citrate, phosphate or formate solution.

80. The method according to claim 72, wherein the step of adding a solution comprising a second stabilizer to the reaction vessel after a given reaction time further comprises adding a cosolvent to the reaction vessel, preferably, the cosolvent is one or more selected from the group consisting of: polysorbate 20, polysorbate 40, polysorbate 60, polysorbate 80, poloxamer 188, polyoxyethylene castor oil, Span, ethanol, propylene glycol, glycerol, polyethylene glycol (average molecular weight of 200-8000), sorbitol, dimethyl sulfoxide, and sodium dodecyl sulfate, further preferably, the cosolvent is polysorbate 80.

81. The method according to claim 72, wherein the step of adding a solution comprising a second stabilizer to the reaction vessel after a given reaction time further comprises adding a chelator for free nuclide to the reaction vessel, and the chelator may be selected from pentetic acid and a salt thereof.

82. The method according to claim 72, wherein it further comprises filtering the radiopharmaceutical aqueous solution through a 0.22 μm filter membrane for sterilization, preferably, the filtration sterilization is performed after adding the solution comprising the second stabilizer.

83. A radiotherapy method for tumors, wherein it comprises administering the pharmaceutical aqueous solution according to claim 64 to a subject in need thereof.
